(19) United States
(12) Patent Application Publication
(10) Pub. No.: US 2021/0139844 A1
(43) Pub. Date: May 13, 2021

(54) REPROGRAMMING FIBROBLASTS TO RETINAL CELLS

(71) Applicant: UNIVERSITY OF NORTH TEXAS HEALTH SCIENCE CENTER, Ft. Worth, TX (US)

(72) Inventors: Sai CHAVALA, Ft. Worth, TX (US); Biraj Mahato, Ft. Worth, TX (US)

(21) Appl. No.: 16/622,823

(22) PCT Filed: Jun. 15, 2018
(86) PCT No.: PCT/US2018/037784

§ 371 Date: Dec. 13, 2019

Related U.S. Application Data
(60) Provisional application No. 62/520,290, filed on Jun. 15, 2017.

(51) Int. Cl. C12N 5/0793; A61K 35/30

(52) U.S. Cl. CPC C12N 5/062; A61K 35/30; C12N 2506/1307; C12N 2501/71; C12N 2501/727; C12N 2501/15; C12N 2506/02; C12N 2501/415; C12N 2501/41; C12N 2501/385; C12N 2503/02; C12N 2506/08; C12N 2501/70

(57) ABSTRACT

Certain aspects of the current invention are directed to a reprogramming of somatic cells to provide therapeutic cells for treatment of diseases such as retinopathies. Certain aspects of the invention are directed to reprogramming compositions, as well as the use of such compositions, for reprogramming somatic cells, the compositions including five small molecules (5C) that can chemically induce conversion to other target cell types.

### CROSS REFERENCE TO RELATED APPLICATIONS

This Application claims priority to U.S. Provisional Patent application Ser. No. 62/520,290 filed Jun. 15, 2017, which is incorporated herein by reference in its entirety.

### FEDERAL SUPPORT PARAGRAPH

This invention was made with government support under EY021171 and EY025667 awarded by National Institutes of Health. The government has certain rights in the invention.

### BACKGROUND OF THE INVENTION

### A. Field of the Invention

The invention generally concerns compositions and methods for reprogramming somatic cells. In particular, the compositions and methods include the reprogramming of somatic cells to cardiomyocytes or retinal cells.

### B. Description of Related Art

Vision loss in most retinal diseases is due to a loss of retinal photoreceptors and/or retinal pigment epithelial cells (RPE). Cell replacement therapy seeks to replace these lost retinal cells in an effort to restore or preserve function. Stem cell therapy is currently at the forefront of cell replacement therapy and has generated a considerable amount of excitement in the field, leading to several biotech startups with several early phase human clinical trials. However, the stem cell approach is limited by political and ethical issues, concern for tumor growth of implanted cells, concern for using of viruses, and concern for host rejection. It would be advantageous to generate retinal replacement cells, as well as other cell types, without the use of stem cells.

### SUMMARY OF THE INVENTION

The compositions of the current invention provide a solution to the problems associated with cell replacement therapy, particularly stem cell therapy. In particular, the current invention provides therapeutic cells based on somatic cells, not stem cells. By way of example, the inventors have discovered a process to reprogram somatic cells, which results in therapeutic cells having appropriate characteristics to treat disease, such as vision loss. Without wishing to be bound by theory, it is believed that the use of the culture conditions described herein results in the reprogramming of somatic cells to a particular target cell type, such as myocytes or retinal cells.

Retinal neuron dysfunction and retinal neuron death is the final common endpoint for blindness in many acquired and inherited retinopathies. Certain aspects of the current invention are directed to a reprogramming of somatic cells to provide therapeutic cells for treatment of diseases, such as retinopathies. Certain aspects of the invention are directed to reprogramming compositions, as well as the use of such compositions, for reprogramming somatic cells, the compositions including five small molecules (5C) that can chemically induce conversion to other target cell types.

In particular aspects, fibroblasts are converted to chemically induced photoreceptor precursor-like cells (CiPPCs), chemically induced photoreceptor cells (ciPRs), chemically induced retinal pigment epithelium cells (ciRPE), or chemically induced retinal ganglion cells (ciRGCs). Collectively, these cells are called chemically induced retinal cells (ciRCs). CiPPCs have a similar transcriptome signature compared to native P5 photoreceptor precursors, and demonstrate functional improvement in RD1 mice, rod photoreceptor degeneration mice, as evidenced by electroretinogram (ERG) and pupillometric (PLR) improvement.

Certain embodiments of the invention are directed to methods of chemically converting a somatic cell to a target cell and the target cells produced by these methods. In certain aspects the target cell is a hepatocyte, cardiomyocyte, hair sensory cell, or retinal cell. The methods can include one or more of (a) culturing the somatic cell in the presence of reprogramming agents converting the somatic cell into a target cell, the reprogramming agents comprising a first reprogramming composition including (i) epigenetic modifier, (ii) glycogen synthase kinase-3 (GSK-3) inhibitor, (iii) TGFβR/ALK5 inhibitor, (iv) cAMP raising compound (e.g., Adenyl cyclase activator); and a second reprogramming composition comprising an enhancing agent, the culturing forming a reprogrammed cell culture; and (b) identifying the target cell in the reprogrammed cell culture. Based on the time of culture various target cells can be identified and isolated. The retinal cell can be a retinal photoreceptor or a retinal pigment epithelial cell.

In certain aspects the epigenetic modifier is a cytochrome P450 2C9 (CYP2C9) inhibitor. The epigenetic modifier can be valproic acid (VPA), 5′-Azacytidine (5′ Aza), 2-(hexahydro-4-methyl-1H-1, 4-diazepin-1-yl)-6,7-dimethoxy-N-[1-(phenylmethyl)-4-piperidinyl]-4-quinazolinamine, trihydrochloride, hydrate (BIX-01294) or a combination thereof. In particular aspects the epigenetic modifier is VPA.

The Glycogen synthase kinase-3 (GSK-3) inhibitor can be Li+, 6-[2-[[4-(2,4-dichlorophenyl)-5-(5-methyl-1H-imidazol-2-yl)pyrimidin-2-yl]amino]ethylamino]-pyridine-3-carbonitrile (CHIR99021), (2′Z,3′E)-6-Bromoindirubin-3′-oxime (BIO), 3-(2,4-Dichlorophenyl)-4-(1-methyl-1H-indol-3-yl)-1H-pyrrole-2,5-dione (SB216763), or a combination thereof. In particular aspects the GSK-3 inhibitor is CHIR99021.

The TGFβR/ALK5 inhibitor can be an inhibitor(s) of Transforming Growth Factor beta (TGFβ) signaling pathway, such as one or more of a Transforming Growth Factor Receptor type I (TGFBR1) kinase inhibitor (e.g., 2-(3-(6-Methylpyridin-2-yl)-1H-pyrazol-4-yl)-1,5-naphthyridine (RepSox), 4-[4-(1,3-benzodioxol-5-yl)-5-(2-pyridinyl)-1H-imidazol-2-yl]benzamide (SB-431542), 3-(6-Methyl-2-pyridinyl)-N-phenyl-4-(4-quinolinyl)-1H-pyrazole-1-carbothioamide (A8301) or combinations thereof) or an anti-TGF-β-antibody, or a nucleic acid agent such as an siRNA. In particular aspects the TGFβR/ALK5 inhibitor is Repsox.

The cAMP raising compound can be an adenyl cyclase activator. In a particular aspect the adenyl cyclase activator is forskolin.

The enhancing agent is an agent or compound that increases the efficiency of production of reprogrammed cells or increase the rate of production of reprogrammed cells. By “increasing the efficiency” of reprogrammed cell production is meant that the percentage of reprogrammed cells in a given population of cells is at least 5, 10, 15, 20, 25, 30, 40, 45, 50, 55, 60, 65, 70, 75, 80, 85, 90, 95% or higher in populations treated with a such an agent than a comparable population of cells not treated or administered the enhancing agent. In certain aspects the efficiency can be 2-fold higher, 5-fold higher, 10 fold higher, 100 fold higher to 1000-fold higher or more than a population not treated with the enhancing agent. By “increasing the rate” of production of reprogrammed cells is meant that the amount of time for the induction of target cells takes less time, e.g., at least 2 days less, 3 days less, 4 days less, 5 days less, 6 days less, 1 week less, weeks less, 3 weeks less or more. In certain aspects it is the type and timing of administration, and length of exposure to the enhancing agent that determines which target cell type is produced in greater quantities. In certain aspects the enhancing agent is a WNT inhibitor, PKC inhibitor, p160ROCK inhibitor, Neurogenic agents, TGFβR inhibitor, MEK1/2 inhibitor, histone deacetylase inhibitor, TGFβR/ALK5 inhibitor, histone methyltransferase inhibitor, and/or GSK-3 inhibitor. In particular aspects the enhancing agent is a WNT inhibitor. The WNT inhibitor can be IWR-1 or XAV939. In another aspect a WNT agonist is CHIR99021, a GSK-3 inhibitor. It is not intuitive to add IWR-1, a WNT antagonist to a VCRF combo because CHIR99021 is a WNT agonist and the logical assumption is that IWR-1 could dampen and negate the WNT agonist activity of CHIR99021. However, the inventors work shows that the addition of both molecules results in generating retinal cells. This occurs because both drugs work to increase Axin2, which goes to the mitochondria and initiates a pathway leading to retinal cell identity. In certain embodiments the enhancing agent(s) is a WNT agonist and a WNT antagonist. In certain instances the WNT inhibitor is IWR-1 or XAV939, and the WNT agonist is CHIR99021. The method can result in an Axin2 that is pharmacologically stabilized resulting in mitochondrial reactive oxidative species production and epigenetic modification leading to a retinal cell type. In a further aspect the enhancing agent can be a combination of WNT agonist and antagonist. The term “WNT antagonist” is used herein to include any molecule that partially or fully blocks, inhibits or neutralizes the signaling of the WNT pathway (e.g., canonical WNT signaling), or partially or fully blocks, inhibits or neutralizes a biological activity of a component of the WNT pathway. WNT antagonists do not necessarily bind WNT. For instance, in certain embodiments WNT antagonists bind one or more other components of the WNT pathway such as one or more FZD receptors. Suitable WNT antagonist molecules include, but are not limited to, fragments and/or amino acid sequence variants of native FZD receptor proteins including soluble FZD receptors, as well as derivatives of soluble Frizzled-related proteins (SFRPs), and derivatives of Ror proteins. Suitable Wnt antagonist molecules further include, but are not limited to, antibodies that specifically bind to one or more FZD receptors and antibodies that specifically bind to one or more WNT polypeptide. Soluble SFRP and Ror receptors are described in US Patent publication 2011/0305695, which is herein incorporated by reference.

In certain aspects the somatic cell is a fibroblast, blood cell, epithelial cell, lung cell, glia, neuron, adipocyte, or hepatocyte. In particular aspects the somatic cell is a fibroblast. In other aspects the somatic cell be an immune cell from the blood or periphery, skin cell (kerantinocyte), or an epithelial cell isolated from the urine. The somatic cell can be a Muller cell or another cell type resident in the retina. Cells resident in the retina include, but are not limited to glial cells, astrocytes, or immune cells.

Certain embodiments are directed to methods that include the steps of culturing the somatic cells by (i) seeding the somatic cells; (ii) exposing the seeded somatic cells to a first reprogramming composition as described herein for a first induction period forming an induced cell population, and exposing the induced cell population to a second reprogramming composition comprising an enhancing agent for a second induction period forming a target cell. In certain aspects the reprogramming composition includes valproic acid, CHIR99021, RepSox, and forskolin. In other aspects the target cell is a retinal cell. The enhancing agent can be a WNT inhibitor, such as IWR-1.

Other embodiments are directed to a hepatocyte, cardiomyocyte, sensory hair cell, or retinal cell produced by the methods described herein. The retinal cell can be photoreceptor, RGC cell, or a RPE-like cell.

Certain embodiments are directed to methods of generating a retinal pigment epithelial cell, the method including (a) culturing the somatic cell in the presence of reprogramming agent converting the somatic cell into a target cell, the reprogramming agents comprising a first reprogramming composition comprising valproic acid, CHIR99021, RepSox, and forskolin, and a second reprogramming composition comprising an enhancing agent, the culturing forming a reprogrammed cell culture; and (b) identifying the target cell in the reprogrammed cell culture.

Other embodiments are directed to methods of generating a photoreceptor cell, the method including (a) culturing a somatic cell in the presence of reprogramming agents converting the somatic cell into a target cell, the reprogramming agents comprising a first reprogramming composition including valproic acid, CHIR99021, RepSox, and forskolin, and a second reprogramming composition comprising an enhancing agent, the culturing forming a reprogrammed cell culture; and (b) identifying the target cell in the reprogrammed cell culture.

Still other embodiments are directed to methods of generating a retinal progenitor cell, the method including (a) culturing a somatic cell in the presence of reprogramming agent converting the somatic cell into a target cell, the reprogramming agents comprising a first reprogramming composition comprising valproic acid, CHIR99021, RepSox, and forskolin, and a second reprogramming composition comprising an enhancing agent, the culturing forming a reprogrammed cell culture; and (b) identifying the target cell in the reprogrammed cell culture.

Certain embodiments are directed to methods of treating a disorder of the eye in a subject in need thereof by delivering to the eye of the subject an effective amount of a retinal cell produced by the methods described herein. The disorder can be retinal atrophy, optic nerve injury, optic nerve atrophy, age-related macular degeneration, inherited retinal degeneration, diabetic retinopathy, sickle cell retinopathy, glaucoma, cystoid macular edema, retinal detachment, vascular occlusion, photoreceptor cell degeneration, infection, vision loss and any combination thereof. In particular aspects the disorder is glaucoma. In certain aspects the disorder can be hearing or hair loss, which can be treated by topical application of one or more reprogramming agents. In another aspect the disorder can be hearing loss, which can be treated by in situ or ex vivo reprograming of a cell to a sensory hair cell in the ear.

Other embodiments are directed to methods of treating a disorder of the eye in a subject in need thereof, comprising delivering to the eye of the subject an effective amount of a combination of valproic acid (V), CHIR99021 (C), RepSox (R), forskolin (F), and an enhancing agent. The combination of small molecules is administered by intraocular injection.

Certain embodiments are directed to methods of using all or a combination of small molecules (valproic acid (V), CHIR99021 (C), RepSox (R), forskolin (F), and IWR1 (I) “VCRFI”; Shh (S), Taurine (T), Retinoic acid (R) “STR”) to differentiate stem or progenitor cells, that may or may not be cultured in a 3D matrix or as a organoid, in a more efficient manner to a retinal lineage.

Other aspects are directed to methods of using all or a combination of small molecules (VCRFI, STR) to rejuvenate an aged cell and make it youthful.

Other methods use all or a combination of small molecules (VCRFI, STR) to restore the function of a damaged, diseased, and/or aged cells.

Further embodiments are directed to methods of generating a retinal ganglion cell, the method comprising: (a) culturing the somatic cell in the presence of reprogramming agent converting the somatic cell into a target cell, the reprogramming agents comprising a first reprogramming composition comprising (i) epigenetic modifier, (ii) glycogen synthase kinase-3 (GSK-3) inhibitor, (iii) TGFβR/ALK5 inhibitor, (iv) cAMP raising compound, and a second reprogramming composition comprising an enhancing agent, the culturing forming a reprogrammed cell culture; and (b) identifying the target cell in the reprogrammed cell culture.

Other aspects are directed to a diagnostic method using a converted cell as an indicator or model cell for testing or screening therapeutic compounds or potential therapeutic compounds. In certain aspects the compounds can be useful for treating a person with neuronal degeneration.

In certain aspects the methods described herein can further include manipulating the somatic cell prior to conversion. In certain aspects manipulating a somatic cell includes gene editing by any well-known method. Gene editing can be performed prior to cell conversion to replace a defective inherited gene prior to therapeutic cell transplantation

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

“Somatic cell,” as used herein, refers to a cell that forms, in part, the body of an organism. Examples of somatic cells include, but are not limited to fibroblasts, keratinocytes, skin cells, blood cells, epithelial cells, lung cells, glia, neurons, adipocytes, and hepatocytes. In certain aspects somatic cells can be isolated and cultured from tissue or biological fluid, including but not limited to biopsy, needle aspirates, blood, urine, and the like.

“Subjects” as used herein include any animal in which treatment of a disorder is necessary or desired, particularly a disorder of the eye. In some embodiments, a subject of this invention can be a mammalian subject, which can be a human subject. Subjects may also include animal subjects, particularly mammalian subjects such as canines, felines, bovines, caprines, equines, ovines, porcines, rodents, lagomorphs, primates (including non-human primates), etc., for veterinary medicine or pharmaceutical drug development purposes.

The terms “therapeutically effective amount” and “effective amount” as used herein are synonymous unless otherwise indicated, and mean an amount of cells or compositions of the present invention that is sufficient to improve the condition, disease, or disorder being treated and/or achieved the desired benefit or goal.

Determination of a therapeutically effective amount, as well as other factors related to effective administration of a composition of the present invention to a subject of this invention, including dosage forms, routes of administration, and frequency of dosing, may depend upon the particulars of the condition that is encountered, including the subject and condition being treated or addressed, the severity of the condition in a particular subject, the particular therapeutic being employed, the particular route of administration being employed, the frequency of dosing, and the particular formulation being employed. Determination of a therapeutically effective treatment regimen for a subject of this invention is within the level of ordinary skill in the medical or veterinarian arts. The amount of active ingredient that can be combined with the carrier materials to produce a single dosage form varies depending upon the subject being treated and the particular mode of administration.

“Treat,” “treating” or “treatment” as used herein refers to any type of action or administration that imparts a benefit to a subject that has a disease or disorder, including improvement in the condition of the patient (e.g., reduction or amelioration of one or more symptoms), delay in the progression of the disease, healing, reversal of the disease or disorder, etc.

Administration of a composition of this invention can be by administration into the eye, for example by injection into the eye (i.e., intraocular injection, which can be for example, intraretinal injection, subconjunctival, suprachoroidal injection, subretinal injection, intracorneal injection, intracameral injection and/or intravitreal injection). In some embodiments, administration may be by implant, via a matrix, via a gel, ointment, liquid drop or any combination thereof.

The terms “genetic modification” and “genetically modified” refer to a permanent or transient genetic change induced in a cell following introduction of a nucleic acid molecule (i.e., a nucleic acid molecule exogenous to the cell). Genetic modification can be accomplished by incorporation of the nucleic acid molecule into the genome of the host cell, or by transient or stable maintenance of the nucleic acid molecule as an extrachromosomal element.

It is further noted that the claims may be drafted to exclude any element of this invention. As such, this statement is intended to serve as antecedent basis for use of such exclusive terminology as “solely,” “only” and the like in connection with the recitation of claim elements, or use of a negative limitation.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or any variation of these terms includes any measurable decrease or complete inhibition to achieve a desired result.

The term “effective,” as that term is used in the specification and/or claims, means adequate to accomplish a desired, expected, or intended result.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1F. Conversion of fibroblast to retinal photoreceptor likes cells (CiPPC) by small molecules: (FIG. 1A) Schematic presentation of mouse reprogramming protocol. Nrl-GFP MEFs were seeded on d0 in IMR90 medium. Small molecules added on d1 and d3 in PIM. V, VPA; C, CHIR99021; R, Repsox; F, Forskolin; S, Shh; T, Taurine; R, Retinoic acid; PIM, photoreceptor induction medium. (FIG. 1B) Fluorescence microscopic images of Nrl-GFP reporter expressing converted cells on d11 (round) and d15 (neuron like). (FIG. 1C) Scheme of human HFL-1-Nrl-DsRed cell reprogramming. IWR1 added on d2 and STR added on d5. (FIG. 1D) Fluorescence micrograph of converted DsRed expressing human photoreceptor like cells at d6 (round) and d8 (neuron like). (FIG. 1E) Number of Nrl-GFP positive cells after conversion protocol (d11) upon treatment with each small molecule alone. (FIG. 1F) Number of Nrl-GFP positive cells upon substation of indicated small molecule from the cocktail.

FIGS. 2A-2H. Characterization of Chemically converted photoreceptor like cells (CiPRLC): (FIG. 2A) Flow sorting of reprogrammed Nrl-GFP positive cells (dot plot). (FIG. 2B) RT-PCR showing mRNA expression of photoreceptor specific indicated genes. (FIG. 2C) Fluorescence micrographs showing that converted (d11) Nrl-GFP positive cells are also Crx positive. (FIG. 2D) Immunofluorescence showing expression of photoreceptor marker Chx10 in converted Nrl-GFP positive cells. (FIG. 2E) Expression of rhodopsin in converted Nrl-GFP positive cells on d15. (FIG. 2F) Expression of Crx in converted DsRed positive human photoreceptor like cells. (FIG. 2G) Lineage tracing, indicating FSP1-tdtomato positive cells are expressing Nrl after conversion on d11. (FIG. 2H) Number of converted Nrl-GFP expressing cells from flow sorted tdTomato positive and negative MEFs.

FIGS. 3A-3C. Transcriptome analysis of converted photoreceptor like cells. (FIG. 3A) Principle component analysis (PCA) for all RNASeq samples showing high similarity of CiPPCs with P4 or P6 photoreceptors isolated from Nrl-GFP mouse retina. (FIG. 3B) Heatmap showing expression of rod but not most of the cone specific genes in chemically reprogrammed cells. (FIG. 3C) Chemically converted cells expressing most retina specific transcription factors and some of retinal progenitor specific genes. Expression of most of the retinal ganglion cell specific gene was absent.

FIGS. 4A-4H. Testing the functionality of converted cells in retinal degeneration (rd1) mice. (FIG. 4A) Timeline for in vivo subretinal injection and functional analysis of CiPPCs after transplantation. (FIG. 4B) Scotopic A wave after transplantation of converted CiPPCs. (FIG. 4C) Scotopic B wave after transplantation of converted mouse cells on P59. (FIG. 4D) Pupil analysis of cell transplanted eyes on P128 with low intensity of light (50 Lux). (FIG. 4E) Quantification of pupil constriction after chemically converted cell transplantation. (FIG. 4F) Integration and survival of GFP expressing chemically converted cells in rd1 retina after 3 months of transplantation. High magnification micrographs (right) showing formation of neural connections. (FIGS. 4G, 4H) Expression of recoverin (recvrn) and rhodopsin (Rho) in chemically converted cells 3 months after transplantation into Rd1 retina.

FIGS. 5A-5G. NF-kB modulated Ascl1 expression mediates CiPRLC reprogramming. (FIG. 5A) Expression of Ascl1 at different time points during reprogramming. qPCR results presented as fold change (2^{-ΔΔCT}) Compared to starting MEF. (FIG. 5B) qPCR showing depletion of Ascl1 in mouse embryonic fibroblasts by shRNA mediated RNAi. (FIG. 5C) No. of Nrl-GFP expressing cells after conversion of Ascl1 knockdown and wild type Nrl-GFP MEFs on d1. (FIG. 5D) NF-kB-Luciferase activity during different time points of chemical reprogramming. NF-kB-Luc-MEFs treated with LPS (10 ng/ml) for 4 hrs was used as positive control. (FIG. 5E) rVista sequence alignment of human and mouse Ascl-1 gene showing highly conserved NF-kB binding sites in downstream of 3′UTR region. (FIG. 5F) Chromatin Immunoprecipitation (ChIP) assay showing binding of NF-kB at Ascl-1 loci. (FIG. 5G) Transient transfection analysis with luciferase reporter fused to Ascl-1 promoter along with 3′ intergenic region on day 8 of reprogramming. Plot shows Ascl-1 is positively regulated by NF-kB through a regulatory sequence located at 3′ end.

FIGS. 6A-6G. Mitochondrial ROS (mROS) generated during chemical conversion activates NF-kB. (FIG. 6A) Accumulation of mROS (MitoSox staining) in chemically converted Nrl-GFP positive photoreceptor cells at d11. (FIG. 6B) MEFs after staining with Mitosox. (FIG. 6C) Fluorometric analysis after MitoSox staining, showing generation of mROS during the course of chemical reprogramming. (FIG. 6D) qPCR showing expression of Tfam (mitochondrial transcription factor) in MEFs after ShRNA mediated knockdown and selection. (FIG. 6E) Quantification for the number of Nrl-GFP expressing cells on d11 after depletion (by mitotempo treatment) or generation (Tfam knockdown) of mROS. (FIG. 6F) Fluorometric measurement of mROS (by mitosox staining) after mitotempo treatment and Tfam knockdown on d11 of chemical conversion. (FIG. 6G) Chromatin Immunoprecipitation assay (ChIP) showing less binding of activated NF-kB on Ascl-1 locus upon treatment with mitosox.

FIGS. 7A-7G. Stabilization and mitochondrial localization Axin2 generates mROS: (FIG. 7A) Western blot showing expression of axin2 at different time points of chemical reprogramming. (FIG. 7B) Micrographs showing mitotracker and axin2 staining in MEFs. No co-localization was found between these staining. (FIG. 7C) Confocal micrographs showing axin2 localization in mitochondria of chemically converted Nrl-GFP positive cells. (FIG. 7D) Western blot showing shRNA mediated knockdown of axin2 in mouse embryonic fibroblasts. (FIG. 7E) Quantification for the number of Nrl-GFP expressing cells after axin2 knockdwn on d11. (FIG. 7F) Fluorometric analysis showing generation of less mROS in axin2 depleted cells during chemical reprogramming compared to wild type control on d8 and d1. (FIG. 7G) qPCR analysis showing axin2 knockdown is associated with decreased Ascl1 expression in reprogramming intermediate on d5 and d8.

FIGS. 8A-8C. Preparation and cloning of Nrl-DsRed DNA construct: (FIG. 8A) Map of Addgene vector pNrl-DsRed from where Nrl promoter and Dsred sequence were digested out by restriction digestion. (FIG. 8B) Strategy for the cloning of Nrl promoter and DsRed into pLenti X1 zeo destination vector. (FIG. 8C) DNA gel showing cloned Nrl and DsRed constructs into destination vector.

FIGS. 9A-9D. Characterization of Chemically reprogrammed mouse and human photoreceptor like cells and lineage tracing. (FIG. 9A) Fluorescence micrographs showing expression of Crx in Nrl-GFP expressing converted cells on d16. (FIG. 9B) Fluorescence micrographs showing expression of rhodopsin in Nrl-Dsred expressing chemically converted human cells. (FIG. 9C) RT-PCR showing expression of photoreceptor specific genes in human photoreceptor like cells. (FIG. 9D) Sorting of Fsp1Cre-tdTomato positive MEFs. Left panel: Dot plot for flow sorting, middle panel: MEFs before sorting, Right panel: MEFs after sorting.

FIG. 10A-10B. Brdu staining of chemically converted photoreceptor like cells: (FIG. 10A) Schematic presentation of Brdu staining protocol during chemical reprogramming of Nrl-GFP MEFs to photoreceptor like cells. (FIG. 10B) Fluorescence micrographs showing Brdu staining on day 11 of chemical reprogramming.

FIG. 11A-11C. NF-kB-Luciferase activity and mROS production during chemical reprogramming. (FIG. 11A) Luciferase activity measurement showing decreased NF-kB activation upon mitotempo treatment and increased NF-kB activity upon Tfam depletion. (FIG. 11B) Luciferase activity showing decreased NF-kB activation in axin2 knockdown MEFs on d8 and d11. (FIG. 11C) Accumulation of mitochondrial ROS (mitosox staining) in each of the cells under fluorescence microscope on d8.

FIG. 12A-12F. ciRGC patchclamp demonstrates neuronal activity. (FIG. 12A) Bright field images of patched mouse ciRGC. (FIG. 12B) Measured resting potential. The holding was at −70 mV. (FIGS. 12C, 12D) Sample traces of current with applied voltage. (FIGS. 12E, 12F) IV relationship in mouse ciRGC (n=4 cells) and human ciRGC (n=3 cells).

FIG. 13A-13F. Conversion of human adult dermal fibroblasts to CiPCs. (A) Modified scheme for human adult dermal fibroblasts reprogramming. (B) qPCR analysis (fold change) of converted CiPCs from HADF, showing increased expression of photoreceptor specific genes. (C) Micrograph of Nrl stained HADF converted CiPCs (left panel). Comparison of conversion efficiency between earlier and modified conversion protocol. (D) Expression of photoreceptor specific genes (Crx and Recoverin) in HADF (from a different source, Coriell Institute) converted CiPCs. (E) Expression of photoreceptor specific genes (Nrl, Recoverin, Rhodopsin) in HADF (from ATCC) converted CiPCs.

FIG. 14A-14B. Conversion of human adult dermal fibroblasts to CiRGC. (A) Human CiRGCs converted from adult dermal fibroblasts. Left d7, Right d8. (B) Realtime qPCR showing expression of RGC specific genes like Brn3a, Brn3b, Isl1 etc on d8.

FIG. 15A-15C. Conversion of mouse primary muller cells to retinal ganglion cells by chemicals. (A) Muller cells before conversion. (B) CiRGC cells after conversion on day3 by chemical cocktail. (C) Real time qPCR analysis showing expression of RGC specific genes like Brn3a, Brn3b, Isl1, Nefl and NeN.

FIG. 16A-16C. Conversion of human primary muller cells to retinal ganglion cells (hCiRGC) by chemicals. (A) human muller cells before conversion. (B) hCiRGC cells after conversion on day3 by chemical cocktail. (C) Real time qPCR analysis showing expression of RGC specific genes like Brn3a, Brn3b, Isl1, Nefl and NeN.

FIG. 17A-17C. Conversion of ES cells to neuron like cells by chemicals (A) Mouse ES cells on day1 before chemical treatment. (B) ES cell derived chemically converted neuron like cells on d6. (C) ES cell derived chemically converted neuron like cells on d7.

### DETAILED DESCRIPTION OF THE INVENTION

Many retinopathies are caused by dysfunctions in retinal neurons like photoreceptor and RGC cells. Loss of these retinal neurons is a common end point of such disorders, which results into severe and permanent vision loss. Some animals, e.g., reptiles, have the capacity to regenerate the retina, but the regeneration potential in mammals is restricted. Research in the past decades has significantly increased our understanding regarding the pathogenesis of retinal disorders (Wright et al. Nature reviews. Genetics 11:273-84, 2010; Bramall et al., Annual review of neuroscience 33:441-72, 2010). However treatment opportunities are currently limited. Regenerative cellular therapy has emerged as a promising tool to manage these retinal neuropathies (Schwartz et al., Lancet 385:509-16, 2015). Currently there are two main sources of these therapeutic cells, one is differentiation from ES (embryonic stem) or iPS (induced pluripotent stem) cells and the other is direct or indirect reprogramming from somatic cells (Mellough et al., Stem cells (Dayton, Ohio) 30:673-86, 2012; Zhong et al., Nature communications 5:4047, 2014; Vierbuchen et al., Nature 463:1035-41, 2010). For example neurons have been shown to convert from fibroblasts by defined transcription factors (Vierbuchen et al., Nature 463:1035-41, 2010). However, introduction of ectopic transgenes limits their therapeutic application.

Recently, small molecule mediated direct or indirect reprogramming has emerged as an alternative method to overcome limitations associated with stem cell differentiation (Hu et al., Cell stem cell 17:204-12, 2015; Li et al., Cell stem cell 17:195-203, 2015). This method has been used to obtain different cell types like neurons, astrocytes, and cardiomyocytes for regenerative therapy (Zhang et al., Cell stem cell 17:735-47, 2015; Tian et al., Cell Rep 16:781-92, 2016; Fu et al., Cell research 25:1013-24, 2015).

Several laboratories have successfully generated retinal neurons like photoreceptor and RGCs from ES and iPS cells by differentiating them in the presence of defined medium (Mellough et al., Stem cells 30:673-86, 2012; Wright, Nature biotechnology 31:712-13; Osakada et al., Nat Protoc 4:811-24, 2009). When transplanted, these ES or iPS derived retinal neurons migrate and integrate into the host retina (Gonzalez-Cordero et al., Nature biotechnology 31:741-47, 2013). Photoreceptor cells can also be obtained from fibroblasts by transcription factor mediated cellular reprogramming (Seko et al., PloS one 7:e35611, 2012). Although these replacement cells can integrate into retina their functional efficiency and efficacy are currently limited (Karl et al., PNAS U.S.A. 105:19508-13, 2008; Chen et al., Cell cycle 8:1158-60, 2009; Venugopalan et al., Nature communications 7:10472, 2016). For example transplantation of photoreceptors in degenerate retina resulted into limited and temporary restoration of ERG (Pearson et al., Nature 485:99-103, 2012). This limitation may be due to the compromised quality of the converted cells or limited migration inside transplanted retina. Furthermore molecular mechanisms underlying direct reprogramming is largely unclear.

These drawbacks are further evidence for needed detailed study of the mechanisms of conversion. Recently it has been shown that mitochondria play an essential role in formation of cardiomyocytes and determination of embryonic and hematopoietic stem cell fate (Crespo et al., Stem cells 28:1132-42, 2010; Vannini et al., Nature communications 7:13125, 2016; Mahato et al., Stem cells 32:2880-92, 2014; Rajendran et al., JBC 288:24351-62, 2013). Mitochondrial function has also been shown to have a role in epidermal hair follicle and adipocyte differentiation (Tormos et al., Cell metabolism 14:537-44, 2011; Anso et al., Nature cell biology, 2017). Additionally mitogenic signaling has been found to be critical for nuclear reprogramming and stem cell lineage commitment (Zhou et al., Cell Rep 15:919-25, 2016; Chandel et al., Nature cell biology 18:823-32, 2016; Shadel and Horvath, Cell 163:560-69, 2015).

The inventors describe herein methods for reprogramming mouse and human retinal neurons like cells (CiPPC) from mouse and human fibroblasts by treatment with five small molecules (5C). This chemically converted photoreceptor like cells can improve retinal function in mouse model having retinal degeneration, as well as be adapted to the production of other target cells. Moreover the inventors describe a molecular mechanism where mitochondria act as a signaling organelle to determine the retinal cell fate.

### A. Reprogramming Somatic Cells

Growing evidence for the plasticity of cell fate has opened the possibility of reprogramming of somatic cells in the laboratory. Reprogramming refers to the conversion of one somatic cell type into another, a process that entails the reinstruction of the gene expression profile of a cell. Reprogrammed somatic cells may be used for cell or tissue therapy such that a patient's own cells or histocompatible cells can be used for treatment of disease or injury.

Each somatic cell type expresses a characteristic repertoire of genes, which may be regulated by environmental cues or factors that cause and/or maintain a particular programmed state through signaling networks that lead to the expression and/or activation/repression of regulatory transcription factors and to epigenetic modifications in DNA/chromatin conformation that determine whether a gene is transcribed or not. By manipulating one or more of these regulatory elements or environments, a cell can be caused to adopt a new differentiated, or reprogrammed, state. Without intending to be limited by theory, the inventors contemplate that somatic cells of a given type contain key regulatory elements which can be sufficient to reprogram another cell to become a cell of that type. Thus, a recipient cell may be reprogrammed by exposure to reprogramming agents/protocols, which include small molecule activators or regulators or mimics of reprogramming agents, or antagonists of inhibitors of reprogramming agents.

In certain embodiments, the reprogramming factors include:

(i) Epigenetic modifiers such as valproic acid (VPA), 5′-Azacytidine (5′ Aza), 2-(hexahydro-4-methyl-1H-1, 4-diazepin-1-yl)-6,7-dimethoxy-N-[1-(phenylmethyl)-4-piperidinyl]-4-quinazolinamine, trihydrochloride, hydrate (BIX-01294) or a combination thereof. In particular aspects the epigenetic modifier is VPA.

(ii) The Glycogen synthase kinase-3 (GSK-3) inhibitor can be Li+, 6-[2-[[4-(2,4-dichlorophenyl)-5-(5-methyl-1H-imidazol-2-yl)pyrimidin-2-yl]amino]ethylamino]-pyridine-3-carbonitrile (CHIR99021), (2′Z,3′E)-6-Bromoindirubin-3′-oxime (BIO), 3-(2,4-Dichlorophenyl)-4-(1-methyl-1H-indol-3-yl)-1H-pyrrole-2,5-dione (SB216763), or a combination thereof. In particular aspects the GSK-3 inhibitor is CHIR99021.

(iii) The TGFβR/ALK5 inhibitor can be an inhibitor(s) of Transforming Growth Factor beta (TGFβ) signaling pathway, such as one or more of a Transforming Growth Factor Receptor type I (TGFBR1) kinase inhibitor (e.g., 2-(3-(6-Methylpyridin-2-yl)-1H-pyrazol-4-yl)-1,5-naphthyridine (RepSox), 4-[4-(1,3-benzodioxol-5-yl)-5-(2-pyridinyl)-1H-imidazol-2-yl]benzamide (SB-431542), 3-(6-Methyl-2-pyridinyl)-N-phenyl-4-(4-quinolinyl)-1H-pyrazole-1-carbothioamide (A8301) or combinations thereof) or an anti-TGF-β-antibody, or a nucleic acid agent such as an siRNA. In particular aspects the TGFβR/ALK5 inhibitor is Repsox.

(iv) cAMP raising compound(s) may be used in the method the invention. The cAMP-raising compound (cAMP elevator) is selected from a cAMP-degrading enzyme inhibitor, a cAMP-phosphodiesterase inhibitor, a cAMP-raising drug, a cAMP-raising hormone, an adenylyl cyclase activator, a cAMP analog, IBMX, GLP-1, GIP, glucagon, forskolin, dibutyryl-cAMP, isoproterenol, or a combination thereof. Analogs of cAMP include 8-pCPT-2-O-Me-cAMP (e.g., 8-(4-chlorophenylthio)-2′-O-methyladenosine 3′,5′-cyclic monophosphate); 8-Br-cAMP (e.g., 8-bromoadenosine 3′,5′-cyclic monophosphate); Rp-cAMPS (e.g., Rp-adenosine 3′,5′-cyclic monophosphorothioate); 8-Cl-cAMP (e.g., 8-chloroadenosine 3′,5′-cyclic-monophosphate); Dibutyryl cAMP (e.g., N6,2′-O-dibutyryladenosine 3′,5′-cyclic monophosphate); pCPT-cAMP (e.g., 8-(4-chlorophenylthio)adenosine 3′,5′-cyclic monophosphate); and N6-monobutyryladenosine 3′,5′-cyclic monophosphate. PDE inhibitors include theophylline (e.g., 3,7-dihydro-1,3-dimethyl-1H-purine-2,6-dione; 2,6-dihydroxy-1,3-dimethylpurine; 1,3-dimethylxanthine), caffeine (e.g., 1,3,7-trimethylxanthine); quercetin dihydrate (e.g., 2-(3,4-dihydroxyphenyl)-3,5,7-trihydroxy-4H-1-benzopyran-4-one dihydrate; 3,3′,4′,5,7-pentahydroxyflavone dihydrate); rolipram (e.g., 4-[3-(cyclopentyloxy)-4-methoxyphenyl]-2-pyrrolidinone); 4-(3-butoxy-4-methoxybenzyl)imidazolidin-2-one; propentofylline (e.g., 3,7-dihydro-3-methyl-1-(5-oxobexyl)-7-propyl-1H-purine-2,6-dione; 3-methyl-1-(5-oxohexyl)-7-propylxanthine); 3-isobutyl-1-methylxanthine (e.g., 3,7-dihydro-1-methyl-3-(2-methylpropyl)-1H-purine-2,6-dione; IBMX; 3-isobutyl-1-methyl-2,6(1H,3h)-purinedione; 1-methyl-3-isobutylxanthine); 8-Methoxymethyl-3-isobutyl-1-methylxanthine (e.g., 8-methoxymethyl-IBMX); enoximone (e.g., 1,3-dihydro-4-methyl-5-[4-methylthiobenzoyl]-2H-imidazol-2-one); papaverine hydrochloride (e.g., 6,7-Dimethoxy 1-veratrylisoquinoline hydrochloride). Activators of adenylate cyclase include forskolin.

(v) Enhancing agents such as WNT inhibitor such as IWR1, XAV-939, ICG-001. In certain aspects, the WNT inhibitor is cardionogen 1, calphostin C, CCT031374 hydrobromide, FH535, ICG-001, iCRT14, IWP-2, IWP-4, IWP-12, IWP-L6, N-(Quinolin-8-yl)-4-(exo-4-aza-3,5-dioxotricyclo[5.2.1.02,6]oct-8-en-4-yl)benzamide (IWR1), JW55, JW67, KY02111, LGK-974, MN64, PNU74654, QS11, TAK715, TC-E5001, WAY316606 hydrochloride, WIKI4, WNT-059 or XAV-939. Enhancing agent can also include—PKC inhibitors (e.g., Go 6983), p160ROCK inhibitors (e.g., Y27632), Neurogenic agents (e.g., ISX-9), TGFβR inhibitor (e.g., SB431542), MEK1/2 inhibitor (e.g., PD0325901), histone deacetylase inhibitor (e.g., suberanilohydroxamic acid (SAHA)), TGFβR/ALK5 inhibitor (e.g., A83-01), histone methyltransferase inhibitor (e.g., BIX01294), and/or GSK-3 inhibitor (e.g., BIO).

A first reprogramming compositions according to the present disclosure may comprise one or more of the following compounds (i) epigenetic modifier, (ii) glycogen synthase kinase-3 (GSK-3) inhibitor, (iii) TGFβR/ALK5 inhibitor, (iv) cAMP raising compound (e.g., Adenyl cyclase activator). In certain aspects the first reprogramming compositions include all four types of inhibitors.

A second reprogramming or enhancing composition will comprise one or more of enhancing agent, such as WNT inhibitor, PKC inhibitor, p160ROCK inhibitor, Neurogenic agent, TGFβR inhibitor, TGFβR/ALK5 inhibitor, MEK1/2 inhibitor, histone deacetylase inhibitor, histone methyltransferase inhibitor, and/or GSK-3 inhibitor, including all combinations thereof.

The target cell can be of any species and may be heterologous to the donor cell, e.g., amphibian, mammalian, avian, with mammalian cells being preferred. Especially preferred target cells include human and other primate cells, e.g., chimpanzee, cynomolgus monkey, baboon, other Old World monkey cells, caprine, equine, porcine, ovine, and other ungulates, murine, canine, feline, and other mammalian species.

1. Culture Conditions

Somatic cells can be seeded in a culture plate. The somatic cells can be seeded on 0.1% gelatin coat and exposed to the first reprogramming agent composition. After 2, 3, 4 5, 6, 7, 8, days or more a first reprogramming agent composition with an enhancing agent is introduced. Certain aspect of the present invention include culture medium and culture conditions for reprograming somatic cells as described herein. Cell culture mediums of the invention can include (i) epigenetic modifier, (ii) glycogen synthase kinase-3 (GSK-3) inhibitor, (iii) TGFβR/ALK5 inhibitor, (iv) cAMP raising compound (e.g., Adenyl cyclase activator); and (v) an enhancing agent wherein the culture medium is effective for reprogramming a somatic cell to a target cell.

In some embodiments of the cell culture medium, the culture medium includes an epigenetic modifier at a concentration from about 50 μM to about 5 mM. In some embodiments, the concentration is from about 100 μM to about 4 mM, from about 200 μM to about 3 mM, from about 500 μM to about 2 mM, from about 250 μM to about 1 mM. In certain aspects the concentration is or is about 500 μM.

In some embodiments of the cell culture medium, the culture medium includes a glycogen synthase kinase-3 (GSK-3) inhibitor at a concentration from about 50 nM to about 1 mM. In some embodiments, the concentration is from about 100 nM to about 500 μM, from about 250 nM to about 250 μM, from about 500 nM to about 50 μM, from about 750 nM to about 10 μM. In particular aspects the concentration is or is about 5 μM.

In some embodiments of the cell culture medium, the culture medium includes a TGFβR/ALK5 inhibitor at a concentration from about 50 nM to about 1 mM. In some embodiments, the concentration is from about 100 nM to about 500 μM, from about 250 nM to about 250 μM, from about 500 nM to about 50 μM, from about 750 nM to about 10 μM. In particular aspects the concentration is or is about 2 μM.

In some embodiments of the cell culture medium, the culture medium includes a cAMP raising compound at a concentration from about 50 nM to about 1 mM. In some embodiments, the concentration is from about 100 nM to about 500 μM, from about 250 nM to about 250 μM, from about 500 nM to about 50 μM, from about 750 nM to about 20 μM. In particular aspects the concentration is 10 μM.

In some embodiments of the cell culture medium, the culture medium includes an enhancing agent at a concentration from about 50 nM to about 1 mM. In some embodiments, the concentration is from about 100 nM to about 500 μM, from about 250 nM to about 250 μM, from about 500 nM to about 50 μM, from about 750 nM to about 20 μM. In particular aspects the concentration is 10 μM.

During cell culture the cells can be treated with a cell culture medium including (i) epigenetic modifier, (ii) glycogen synthase kinase-3 (GSK-3) inhibitor, (iii) TGFβR/ALK5 inhibitor, and (iv) cAMP raising compound for a period ranging from 2 days to 8 days, in particular about 3 days. After 2 to 8 days the enhancing agent is added to the culture medium for a period ranging from 9, 10, 11, 12, 13, 14, 15, 16, 17, 18, 19, to 20 days. The cell culture medium can be changed regularly until a reprogrammed cell forms.

In addition to the reprograming agents described herein, the culture medium can be any cell culture medium commonly used in the art. For example, the culture medium generally includes saline. An example of cell culture medium includes saline, a pH of 7.4 PBS, DMEM medium, or fibroblast basic medium (FBM, Lonza). In some embodiments, the culture medium can include additional components or agents.

As used herein, the term “sufficient time” shall mean a period sufficiently long to reprogram the mammalian cell by the culture medium disclosed herein. In some embodiments, the term “sufficient time” ranges from hours to days. Sufficient time can include 8, 12, 16, 20, 24 hrs to about 2, 3, 4, 5, 6, 7, 8, 9, 10, 11, 12, 13, 14, 15, 16, 17, 18, 19, 20 days. In particular aspects a time sufficient for reprogramming is from about 9 days to about 20 days in culture under reprogramming conditions as described herein.

In some embodiments, the method provided herein further includes changing culture medium with fresh culture medium regularly. The term “regularly” shall mean changing culture medium hourly, bi-hourly, four times a day, twice a day, daily, once per two-day, bi-weekly, weekly, bi-monthly, or monthly.

### B. Treatment of Disease

Many diseases resulting from the dysfunction of cells may be amenable to treatment by the administration of reprogrammed cells. These include diseases of cardiac, neurological, endocrinological, vascular, retinal, dermatological, and muscular-skeletal systems, and other diseases. A patient's own cells can be transformed or converted into a desired cell type that needs replacement. Thus, reprogramming permits the generation of autologous, genetically matched cells that would not be subject to immune rejection on transplantation. Additionally, reprogrammed cell lines created according to the methods described herein can be a source of cells for transplantation.

In some embodiments of the method, optionally in combination with any or all of the above various embodiments, the disorder is a retina disease, a trauma and injury to a tissue, a skeletal disorder, an organ disease or an injury to skin, muscle, cartilage, tendon, peripheral nerve, spinal cord, blood vessels, or bone.

Preferably the cells are histocompatible with the individual recipient, such that the undesirable use of immunosuppression is decreased or eliminated. For example, histocompatible cells may be obtained from the patient, from a donor related to the patient, or an unrelated donor. Optionally the cells can be genetically modified to alter their histocompatibility profile, such that they are more compatible with the patient.

Among reprogrammed cells that can be produced by these methods are such sought-after cells as cardiomyocytes, neurons, oligodendrocytes, retinal pigment epithelium, ganglion cells, photoreceptors, insulin-producing cells, skeletal myoblasts, smooth muscle cells, hepatocytes, and others. Such cells and tissues would satisfy an unmet medical need for tissue and organ repair and could be generated to decrease the risk of immune rejection either through banking a variety of genetically diverse cell lines or via patient-specific reprogramming. The cells may be used in various methods known in the art, including being injected into a patient or organ, grown on a scaffold and surgically implanted, directly applied to the site of an injury, etc.

A reprogrammed somatic cell of this invention can be used to treat a subject in need of such treatment. Similarly, an induced RPE, induced PR cell, and/or induced retinal progenitor cell of this invention can be used to treat a subject in need of such treatment. A cell of this invention can be introduced into a recipient subject (e.g., a subject in need of treatment), where introduction of the cell(s) into the subject treats a condition or disorder in the subject. Thus, in some embodiments, a method of treatment involves administering to a subject in need thereof a population of reprogrammed somatic cells of this invention. In some embodiments, a method of treatment of this invention involves administering to a subject in need thereof a population of induced RPEs, induced PR cells, induced ganglion cells, and/or induced retinal progenitor cells of this invention. The cells can be from the subject or the cells can be from an individual other than the subject.

In some embodiments, the present disclosure provides a method for performing cell transplantation in a recipient subject in need thereof, the method generally involving; (i) generating an induced RPE, an induced PR cell, induced retinal ganglion cell, and/or an induced retinal progenitor cell from a somatic cell obtained from a donor, wherein the donor is immunocompatible with the recipient subject; and (ii) transplanting one or more of the induced cells of this invention into the recipient subject. In some embodiments, the recipient subject and the donor are the same individual. In some embodiments, the recipient subject and the donor are not the same individuals.

In some embodiments, the present disclosure provides a method for performing cell transplantation in a recipient subject in need thereof, comprising; (i) reprogramming a somatic cell, wherein the somatic cells are obtained from a donor, wherein the donor is immunocompatible with the recipient subject; and (ii) transplanting one or more of the reprogrammed somatic cells into the recipient subject. In some embodiments, the recipient subject and the donor are the same individual. In some embodiments, the recipient subject and the donor are not the same individuals.

The present disclosure provides methods for treating a disorder of the eye in an individual, comprising administering to a subject in need thereof a therapeutically effective amount of: (a) a population of induced RPEs, a population of induced PR cells, induced retinal ganglion cells, and/or a population of induced retinal progenitor cells prepared according to the methods of this invention; and/or (b) a population of reprogrammed somatic cells prepared according to the methods of this invention.

Non-limiting examples of a disorder of the eye that can be treated according to the methods of this invention include retinal atrophy, optic nerve injury, optic nerve atrophy, age-related macular degeneration, inherited macular degeneration, diabetic retinopathy, sickle cell retinopathy, glaucoma, cystoid macular edema, retinal detachment, vascular occlusion, photoreceptor cell degeneration, infection, vision loss and any combination thereof.

For administration to a mammalian subject, a population of induced RPEs induced PR cells, induced retinal progenitor cells and/or a population of genetically modified somatic cells, generated using methods of the present invention can be formulated as a pharmaceutical composition. A pharmaceutical composition can be a sterile aqueous or non-aqueous solution, suspension or emulsion, which additionally comprises a physiologically acceptable carrier (i.e., a non-toxic material that does not interfere with the activity of the cells). Any suitable carrier known to those of ordinary skill in the art may be employed in a pharmaceutical composition of this invention. The selection of a carrier will depend, in part, on the nature of the substance (i.e., cells or chemical compounds) being administered.

The present disclosure provides methods for treating other disorders as well, such as hearing loss and hair loss, comprising administering to a subject in need thereof a therapeutically effective amount of: (a) a population of induced sensory hair cells or a population of induced progenitor sensory hair cells prepared according to the methods of this invention; and/or (b) a population of reprogrammed somatic cells prepared according to the methods of this invention. In other aspects, target somatic cells can be treated in situ using the treatment methods described herein.

Hearing Loss. The cochlear sensory epithelium contains hair cells adapted for the detection of sound, which is transduced by stereocilia at their apical surfaces. Hair cells produced during development are post-mitotic and are not replaced after loss or as part of normal cell turnover in mammals. As a result, deafness due to hair cell loss is irreversible. Hair cell development during the embryonic period includes a complex series of fate decisions, in which prosensory epithelial cells acquire different fates, either hair cell or supporting cell. Certain aspects of the methods described herein can be used to regenerate cochlear hair cells in adult animals that correlated with recovery of hearing after noise-induced hearing loss. Thus, in one aspect the invention features methods for treating hearing loss caused by loss of cochlear hair cells in a post-neonatal mammal. The methods include systemically or locally administering to the ear of the mammal a composition comprising a therapeutically effective amount or regimen of reprogramming factors or reprogrammed cells, wherein the therapeutically effective amount is an amount sufficient to restore hearing at one or more frequencies.

In addition, the compositions and methods featured herein can be used prophylactically, such as to prevent, reduce or delay progression of hearing loss, deafness, or other auditory disorders associated with loss of hair cells.

In general, the compounds and methods described herein can be used to generate hair cell growth in the ear and/or to increase the number of hair cells in the ear (e.g., in the inner, middle, and/or outer ear). For example, the number of hair cells in the ear can be increased about 2-, 3-, 4-, 6-, 8-, or 10-fold, or more, as compared to the number of hair cells before treatment. This new hair cell growth can effectively restore or establish at least a partial improvement in the subject's ability to hear. For example, administration of an agent can improve hearing loss by about 5, 10, 15, 20, 40, 60, 80, 100% or more.

Hair Loss. Other embodiments include the treatment of hair loss by reprogramming cells of the hair follicle. The mammalian hair fiber is composed of keratinized cells and develops from the hair follicle. The hair follicle is a peg of tissue derived from a downgrowth of the epidermis, which lies immediately underneath the skin's surface. The distal part of the hair follicle is in direct continuation with the external, cutaneous epidermis. Although a small structure, the hair follicle comprises a highly organized system of recognizably different layers arranged in concentric series. Active hair follicles extend down through the dermis, the hypodermis (which is a loose layer of connective tissue), and into the fat or adipose layer.

At the base of an active hair follicle lies the hair bulb. The bulb consists of a body of dermal cells, known as the dermal papilla, contained in an inverted cup of epidermal cells known as the epidermal matrix. Irrespective of follicle type, the germinative epidermal cells at the very base of this epidermal matrix produce the hair fiber, together with several supportive epidermal layers. The lowermost dermal sheath is contiguous with the papilla basal stalk, from where the sheath curves externally around all of the hair matrix epidermal layers as a thin covering of tissue. The lowermost portion of the dermal sheath then continues as a sleeve or tube for the length of the follicle.

Certain aspects of the methods described herein can be used to regenerate germinative epidermal cells for recovery from hair loss. Thus, in one aspect the invention features methods for treating hair loss caused by loss of germinative epidermal cells in a mammal. The methods include systemically or locally administering to the scalp or hair follicle of the mammal a composition comprising a therapeutically effective amount or regimen of reprogramming factors or reprogrammed cells, wherein the therapeutically effective amount is an amount sufficient to restore the capability to grow hair.

### C. Methods of Identifying and Verifying Target Cells.

Reprogrammed cells can be identified and verified using various methods. These methods include examining cell and colony morphology; determining whether the cells exhibit functional characteristics of the target cell type; determining whether cells express characteristic markers of the target cell type; and comparing gene methylation to the target cell type.

Additionally, candidate reprogrammed cells can be analyzed to determine whether unwanted genetic and/or epigenetic alterations are present. For example, cells may be karyotyped, such as by cytological methods (including classic and spectral karyotyping methods) and/or by sequencing-based methods (e.g., digital karyotyping). Cells can also be tested to determine whether loss of heterozygosity has occurred, for example by comparing the genome-wide SNP profile between untreated cells and reprogrammed cells, with loss of heterozygosity indicating that potentially undesired recombination events have occurred. Cells can also be tested to detect aberrant expression of oncogenes and/or tumor suppressors. Cells can also be tested for unwanted genome sequence modification by partial or full genome sequencing, which is optionally targeted to the sequences of particular genes (e.g., genes involved in growth regulation). Cells can also be tested for undesired epigenetic changes, such as undesired histone modification.

The present invention additionally provides an induced retinal pigment epithelium cell, an induced photoreceptor cell, retinal ganglion cell, and/or an induced retinal progenitor cell produced by the respective methods of this invention. Also provided herein is a population of induced retinal pigment epithelium cells, a population of induced photoreceptor cells and a population of induced retinal progenitor cells produced by the respective methods of this invention.

Additional aspects of this invention provide methods of treating a disorder of the eye in a subject (e.g., a subject in need thereof), comprising delivering to the eye(s) of the subject an effective amount of a cell of this invention.

In the methods of this invention, the reprogrammed target cells can be analyzed for characteristics of endogenous retinal pigment epithelium cells, endogenous photoreceptor cells, or endogenous retinal progenitor cells, respectively. Non-limiting examples of characteristics of an endogenous retinal pigment epithelium cell include gene and protein expression of RPE65, Cralbp, Bestrophin, tyrosinase, resting membrane and transepithelial potential, polarized secretion of VEGF and PEDF, and phagocytosis. Non-limiting examples of characteristics of an endogenous photoreceptor cell include gene and protein expression of rhodopsin, recoverin, peripherin, converting light stimulus into an electrical impulse. Non-limiting examples of characteristics of an endogenous retinal progenitor cell include the ability to differentiate into retinal neuronal subtypes, gene and protein expression of Nestin, Sox2, ChxlO, Pax6, Six6, Six3, or Rax.

### D. Pharmaceutical Compositions and Administration

The present invention provides a composition comprising a reprogrammed somatic cell (e.g., an induced RPE, induced PR cell and induced retinal progenitor cell; progeny of an induced RPE, induced PR cell and induced retinal progenitor cell) and a suitable carrier. A composition of this invention can comprise a reprogrammed cell of this invention and can in some embodiments comprise one or more additional components, which components are selected based in part on the intended use of the reprogrammed cell of this invention. Suitable components include, but are not limited to, salts; buffers; stabilizers; protease-inhibiting agents; cell membrane- and/or cell wall-preserving compounds, e.g., glycerol, dimethylsulfoxide, etc.; nutritional media appropriate to the cell; and the like.

In some embodiments, a composition of this invention can comprise a reprogrammed cell of this invention and a matrix or support, where a reprogrammed cell of this invention is associated with the matrix. The term “matrix” refers to any suitable carrier material to which the reprogrammed cells are able to attach themselves or adhere in order to form a cell composite. In some embodiments, the matrix or carrier material is present already in a three-dimensional form desired for later application.

For example, a matrix (also referred to as a “biocompatible substrate”) can be a material that is suitable for implantation into a subject. A biocompatible substrate does not cause toxic or injurious effects once implanted in the subject. In one embodiment, the biocompatible substrate is a polymer with a surface that can be shaped into a desired structure or part of a desired structure. The biocompatible substrate can provide a supportive framework that allows cells to attach to it and/or grow on it. Suitable matrix components include, but are not limited to collagen; gelatin; fibrin; fibrinogen; laminin; a glycosaminoglycan; elastin; hyaluronic acid; a proteoglycan; a glycan; poly(lactic acid); poly (vinyl alcohol); poly(vinyl pyrrolidone); poly(ethylene oxide); cellulose; a cellulose derivative; starch; a starch derivative; poly(caprolactone); poly(hydroxy butyric acid); mucin; and the like. A reprogrammed cell/matrix composition of this invention can further comprise one or more additional components, wherein suitable additional components include, e.g., a growth factor; an antioxidant; a nutritional transporter (e.g., transferrin); a polyamine (e.g., glutathione, spermidine, etc.); and the like. The cell density in a reprogrammed cell/matrix composition of this invention can range from about 10² cells/mm³ to about 10⁹ cells/mm³; from about 10² cells/mm³ to about 10⁴ cells/mm³; from about 10⁴ cells/mm³ to about 10⁶ cells/mm³; from about 10⁶ cells/mm³ to about 10⁷ cells/mm³, from about 10⁷ cells/mm³ to about 10⁸ cells/mm³, or from about 10⁸ cells/mm³ to about 10⁹ cells/mm³.

A composition of this invention can include a pharmaceutically acceptable carrier. Suitable carriers include, for example, water, saline, dextrose, glycerol, ethanol, or the like, and combinations thereof. In addition, if desired, the carrier can contain minor amounts of auxiliary substances such as wetting or emulsifying agents or pH buffering agents. Actual methods of preparing such compositions and/or formulations are known, or will be apparent, to those skilled in the art. See, e.g., Remington's Pharmaceutical Sciences. Mack Publishing Company, Easton, Pa., latest edition.

Pharmaceutically acceptable excipients, such as vehicles, adjuvants, carriers or diluents, are well known in the art. Moreover, pharmaceutically acceptable auxiliary substances, such as pH adjusting and buffering agents, tonicity adjusting agents, stabilizers, wetting agents and the like, are well known in the art.

Representative carriers include physiological saline solutions, gelatin, water, alcohols, natural or synthetic oils, saccharide solutions, glycols, injectable organic esters such as ethyl oleate or a combination of such materials. Optionally, a pharmaceutical composition may additionally contain preservatives and/or other additives such as, for example, antimicrobial agents, anti-oxidants, chelating agents and/or inert gases, and/or other active ingredients.

A unit dosage form of an induced RPE population, an induced PR cell population or a retinal progenitor cell population of this invention can contain from about 10,000 to about 10,000,000 cells.

An induced cell population of this invention and/or a population of reprogrammed somatic cells of this invention can be cryopreserved according to routine procedures. For example, cryopreservation can be carried out on from about one to ten million cells in “freeze” medium which can include a suitable proliferation medium, 10% BSA and 7.5% dimethylsulfoxide. Cells are centrifuged. Growth medium is aspirated and replaced with freeze medium. Cells are resuspended as spheres. Cells are slowly frozen, by, e.g., placing in a container at −80° C. Cells are thawed by swirling in a 37° C. bath, resuspended in fresh proliferation medium, and grown as described herein.

In the methods described above, the cell in the eye of the subject can be, but is not limited to, a fibroblast, a retinal neuron, an RPE cell, a PR cell, retinal ganglion cell, a Muller glia cell and any combination thereof. A composition of this invention can be administered to a subject at or near a treatment site in the eye.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Chemical Reprogramming of Fibroblasts to Retinal Cells

### A. Results

Identification of small molecules for derivation of photoreceptor-like cells (CiPPC). To identify small molecules responsible for retinal neuron specific fate, the inventors followed previous studies describing a set of small molecules that can convert fibroblasts into neurons (Hu et al., Cell stem cell 17:204-12, 2015; Zhang et al., Cell stem cell 17:735-47, 2015; Fu et al., Cell research 25, 2015; Cheng et al., Cell research 25:1269-72, 2015). Four out of six small molecules described in these studies, Valproic acid (V), CHIR(C), Repsox (R) and Forskolin(F), are able to convert fibroblast cells into mixed progenitor stage (Fu et al., Cell research 25, 2015). The inventors have exploited this phenomenon to find out additional small molecules those might be important for the determination of retinal neuron specific fate. An array of small molecule along with different medium compositions and time points has been tested as possible candidates. Mouse Embryonic Fibroblasts (MEFs) from a photoreceptor specific reporter mouse (Nrl-GFP) are used as starting cells for the reprogramming studies (Akimoto et al., PNAS 103:3890-95, 2006). If converted to photoreceptors, these MEFs would have GFP expression from the Nrl promoter. After several failures with many small molecules and culture medium the inventors identified a Wnt/0-catenin inhibitor (IWR1) that can covert GFP negative MEFs into GFP⁺ positive round shaped photoreceptor like cells in presence of VCRF between d10 to d11. This conversion requires three additional factors Sonic Hedgehog (S), Taurine (T) and Retinoic acid (R) introduced on day 8 (d8) (FIG. 1A and FIG. 1B). Human fibroblasts (HFL1, ATCC CCL153) can also be converted into photoreceptor like cells by these small molecules. The inventors first generated a HFL-1 cell line having a lentivirally transduced DsRed reporter downstream of Nrl promoter. Drug selected HFL1-Nrl-DsRed cells were used for the reprogramming by the previously described mouse specific protocol with some modifications (FIG. 1C). Round shaped and neuron like Nrl promoter driven DsRed positive cells were evident starting from d6 to d8 (FIG. 1D). Modifications of the method by changing IWR1 concentration and time of its introduction are essential for obtaining the cells. Addition of BDNF, GDNF and NT3 during reprogramming helped these converted human photoreceptor like cells to have a healthy morphology. The inventors have tested the importance of each small molecule in the reprogramming process by using Nrl-GFP MEFs as starting cells. Each of them alone failed to turn on the GFP reporter indicating synergy between the small molecules is crucial for the process (FIG. 1E). The inventors then subtracted each of the small molecules from the cocktail and found that removal of Repsox, Forskolin, CHIR and IWR1 have worst effect on reprogramming process. These results clearly demonstrated that small molecule cocktail (5C) can covert fibroblasts cells into retinal photoreceptor like cells. FIG. 12A-12F illustrates that the ciRGC have neuronal activity.

Characterization of chemically reprogrammed photoreceptor like cells (CiPPC). For further characterization converted GFP⁺ cells were flow sorted on d11 and analyzed for gene expression by RT-PCR (FIG. 2A). Expression of early photoreceptor markers like Otx2, Crx, Nrl and Chx10 was evident in chemically reprogrammed cells (FIG. 2B). Furthermore immunostaining of these round shaped GFP positive cells at d11 showed expression of Chx10, CD73 and Crx (FIG. 2C and FIG. 2D). These results indicated that round shaped GFP positive cells at d11 are putative photoreceptor like cells (CiPPC). When cultured in differentiation medium for additional 5 days, these round shaped cells showed neuron like structure with GFP reporter expression from Nrl promoter. These neuron like GFP⁺ cells were Rhodopsin and Crx positive (FIG. 2E and FIG. 9A). During differentiation process these neuron cells died rapidly.

The inventors then checked the gene expression status of converted human photoreceptor like cells by RT-PCR and immunostaining. DsRed expressing photoreceptor like cells was found to have Crx, Rhodopsin, Chx10 and Otx2 expression (FIG. 2F, FIG. 9B, FIG. 9C).

Fibroblasts origin of initial MEFs were confirmed by using a Cre-LoxP lineage tracing system to follow the fate of original fibroblasts expressing a fibroblast specific marker, Fsp1. At first FSP1-Cre animals were crossed with R26-f-STOP-f-Tdtomato animals and resulting FSP-1-Cre-f-STOP-f-Tdtomato embryos (E12.5) were used for MEF isolation (Fu et al., Cell research 25:1013-24, 2015). These MEFs showed Tdtomato expression in Cre expressing cells. Flow sorted Tdtomato expressing cells were then used for chemical reprogramming. Chemically converted cells on d11 found to have both tdTomato and Nrl expression. Co-localization of tdTomato with the Nrl suggests that Nrl positive cells are truly originated from the fibroblasts (FIG. 2G). It was also found some GFP expressing converted cells from tdTomato negative population after sorting (FIG. 2H). These results demonstrated that chemically converted Nrl positive cells are truly originated from fibroblasts.

CiPPC generation is direct, without a progenitor or proliferative stage. Next the inventors studied whether chemical reprogramming process is direct or through an intermediate progenitor stage. Sox2-GFP MEFs (sox2 is retinal progenitor marker) were used as starting cells for the reprogramming by the chemical cocktail. No reporter expression was found at any stage of the reprogramming process (data not shown). The inventors also have tested whether the reprogramming is passed through an intermediary proliferative stage. To test this, Nrl-GFP MEFs were treated with 5-bromodeoxyuridine (BrdU) with the small molecule induction throughout the culture period for chemical reprogramming. Majority of the GFP+ cells (about 95%) were negative for BrdU, indicating a direct reprogramming (FIG. 10A and FIG. 10B).

CiPPCs possess gene expression signature like rod photoreceptors. Next the inventors studied the detail gene expression signature of these chemically converted cells. The inventors were curious about two aspects, first how close the cells are to their native counterparts; and second, how gene expression signature changes upon treatment with chemicals at the intermediary stages of reprogramming from MEFs to photoreceptor like cells. To address the first aspect the gene expression profiling of chemically converted cells (CiPPCs) with GFP+ rod photoreceptor cells isolated from P3 and P5 pups of Nrl-GFP reporter mice (positive control) were compared (Pearson et al., Nature 485:99-103, 2012; Kim et al., Cell Rep 17:2460-73, 2016; Barbera et al., PNAS 110:354-59, 2013). To address the second aspect two intermediate stages of reprogramming were included at d5 and d8 (RepInterm d5 and RepInterm d8). Additionally transcription profiling of starting MEFs during transcriptome analysis was included. Sanity plot for the RNASeq data showed more than 20 million reds for each sample indicating that the data was clean and there was negligible batch effect (FIG. 10C). Unsupervised Pearson's correlation analysis (PCA) of averaged normalized FPKM values showed a shift of CiPRs transcriptome towards P4 and P6 Nrl-GFP⁺ cells. Little similarity was found from cells isolated from P3 pups. Reprogrammed cells found to have no similarity with MEFs and reprogramming intermediates at d5 and d8. PCA analysis also indicated a transcriptome shifting of reprogramming intermediates from the starting MEFs (FIG. 3A).

PCA data was further validated by heatmap analysis for expression of known genes. Chemically reprogrammed cells were found to have high expression of Rod specific genes but other retinal neurons like cone, ganglionic, amacrine, horizontal specific genes were expressed at very low level (FIG. 3B, FIG. 3C and data not shown). Moderate expression of some bipolar and Muller cell specific genes were also evident. When searched for retinal progenitor (RPC) specific gene expression in converted cells a very low level of expression for RPC specific genes except Vsx2 and Notch1 was found (FIG. 3C). These chemically reprogrammed cells were found to have high expression of retina specific transcription factors like Otx2, Nrl, Crx etc confirming its retinal identity (FIG. 3C). When the inventors checked the expression of other homeobox and blH transcription factors, very low level of expression except neurod1 and neurod4 were found. Notably some of the transcription factors which have a reported role in photoreceptor specification like Rorb, Ascl1, Pias3, Thrb, Rxrg are induced in d5 and d8 intermediates (FIG. 3C). These results clearly indicated that chemically converted photoreceptor like cells have similar gene expression profiling like their native counterpart isolated from reporter mice.

Transplantation and functional testing of reprogrammed cells in photoreceptor deficient rd1 mice. To test the functionality of these chemically converted photoreceptor like cells inside the mouse retina, flow sorted GFP⁺ cells were transplanted sub-retinally into retinal degeneration mice (Rd1) and analyzed for electroretinogram (ERG) and Pupillometry. Rd1 is a clinically relevant mouse model of inherited retinopathy, Retinitis Pigmentosa (Barbera et al., PNAS 110:354-59, 2013). These mice undergo progressive loss of photoreceptors with nearly complete loss within three weeks. About 100K reprogrammed Nrl-GFP positive cells were transplanted subretinally into 6 eyes of Rd1 mice on d31. PBS injected eyes were used as control. Cell transplanted and PBS injected mice were analyzed for Scotopic A and B wave analysis on d45 and d59 (FIG. 4A). At d45 three of the eyes showed improved scotopic A wave compared to PBS injected control (FIG. 4B). Although these ERG values were lower than the wild type control. The inventors did not observe any detectable Scotopic B-wave and Photopic B-wave at this time point. On day 59 only one eye showed significant increase of Scotopic B-wave (FIG. 4C). The inventors have performed similar ERG analysis on day 90 but did not find any electrical response from any of the eyes (data not shown). Previous reports suggest that despite robust integration of cells during subretinal injections scotopic ERG responses may not be evident, albeit such responses can be readily recorded in wild type animals (Pearson et al., Nature 485:99-103, 2012; Lamba et al., Cell stem cell 4:73-79, 2009). One recent report indicated that Pupillary Light Response (PLR) may be preserved at late time points (3 months) when there was nearly undetectable A wave and trace amount of B-wave present (Zhu et al., Cell stem cell, 2016). These observations motivated the inventors to test for PLR in cell transplanted animals. Light mediated pupil constriction depends on photoreceptors sending light stimuli via functional connections with the inner retina to midbrain nuclei and back to pupillary muscles. The inventors measured the pupil constriction and found about 30% increase in pupil constriction in photoreceptor transplanted eyes compared to PBS control (FIG. 4D and FIG. 4E). Finally, cryo-sectioned CiPPC injected retinas were tested for the existence of GFP positive photoreceptor like cells. No nuclear layer (ONL) was evident after 3 months in transplanted retina, indicating complete retinal degeneration. Transplanted cells were present in existing inner nuclear layer and subretinal space. These cells also formed connections with each other and part of retina (FIG. 4F). Nrl-GFP⁺ cells inside retina were positive for photoreceptor markers OTX2, Rhodopsin and recoverin (FIG. 4G and FIG. 4H). These results clearly demonstrated that chemically reprogrammed Nrl-GFP⁺ cells survived, migrated, and differentiated inside photoreceptor deficient (rd1) retina.

NF-kB induced ASCL-1 expression mediates CiPPCs reprogramming. Next the inventors looked at the mechanism of chemical reprogramming. Previous reports demonstrated that overexpression of proneural transcription factor ASCL-1 can reprogram Muller glial cells into retinal neurons both in vitro and in vivo (Brzezinski et al., Development 138:3519-31, 2011; Ueki et al., PNAS 112:13717-22, 2015). RNA Seq analysis showed increased expression of Ascl-1 starting from d8 of reprogramming intermediates (FIG. 3C). The inventors have validated the RNASeq data by qPCR and found a similar trend of Ascl-1 expression (FIG. 5A). Ascl1 induction was not evident during treatment with small molecules alone or in any combinations like VCRF. So the appearance of Ascl-1 expression during conversion was truly due to the combination of chemical cocktail (5C). Recently photoreceptor specific gene expression was reported in Ascl-1 overexpressing cells in vitro and in vivo (Ueki et al., PNAS 112:13717-22, 2015). Moreover Ascl1 has been reported to be expressed transiently in some photoreceptor precursor cells (Swaroop et al., Nat Rev Neurosci 11:563-76, 2010). Depending on these observations the inventors contemplate that Ascl1 might play a role in CiPPCs reprogramming. To test this the inventors have depleted Ascl1 in MEFs from Nrl-GFP reporter mice by shRNA technology (FIG. 5B). Puromycin selected MEFs (for 3 days) were then used for the Chemical conversion. Indeed, it was found that there were 70-80% less generation of GFP+ round photoreceptor like cells on day 11 compared to control shRNA (FIG. 5C).

Next the inventors investigated the cause of ASCL-1 induction. In earlier studies, transcription factors like NF-kB, have been employed in early differentiation of neural stem cells and embryonic neurogenesis (Zhang et al., Stem cells 30:510-24, 2012). NF-kB acts as rapid acting primary transcription factor against different cellular stimuli. It is present in the cell in an inactive state and does not require new protein synthesis in order to be functional. This allows NF-kB to be first responder against cellular stimuli. So it was reasoned that NF-kB or any other transcription factor might be involved in upstream of Ascl-1 to induce its expression. NF-kB activation was checked during chemical conversion of MEFs transduced with NF-kB-luciferase construct. The inventors found activation of NF-kB starting from d5 and reaching highest at d11 (FIG. 5D). These results indicated that NF-kB might be involved upstream of Ascl1 to induce its expression. To confirm the role of NF-kB in Ascl-1 induction bioinformatics (rVista) was performed followed by quantitative ChIP assay (FIG. 5E). One of the sites located near to 3 prime UTR of Ascl-1 coding regions showed positive binding for NF-kB (FIG. 5F). Furthermore transient transfection analysis with a luciferase reporter gene confirmed that NF-kB positively regulates Ascl1 by binding to its 3′ intergenic sequence on day 8 of reprogramming (FIG. 5G). These results clearly indicated that small molecule treatment causes NF-kB activation which in turn binds to Ascl-1 regulatory regions and control its expression.

Mitochondrial ROS activates NF-kB which controls CiPPC reprogramming by retrograde signaling. Next the inventors investigated the reasons behind NFkβ activation. Known inducers of NF-kB are highly variable including TNFα, LPS, Ionizing radiation and mitochondrial ROS (Formentini et al., Mol Cell 45:731-42, 2012; Andreakos et al., Blood 103:2229-37, 2004). Recent reports demonstrated that mitochondria generated ROS may induce a retrograde response to the nucleus through activation of NF-kB (Formentini et al., Mol Cell 45:731-42, 2012). So the inventors contemplated that mitochondrial ROS generated by small molecule treatment may activate NF-kB. To prove this the inventors first measured the mitochondrial ROS at different time points during reprogramming by fluorometry as well as microscopy. The inventors found increased accumulation of mitochondrial ROS indicator mitosox during reprogramming starting from d5 compared to control MEFs (FIG. 6A, FIG. 6B and FIG. 6C). To measure the importance of mROS during chemical reprogramming, conversion experiments were performed in the presence of mitochondrial ROS scavenger, mitotempo. Scavenging of mROS during chemical reprogramming significantly decreased yield of reprogrammed cells (20%) (FIG. 6E). The inventors have confirmed the decrease of mROS generation upon treatment with mitotempo by mitoSox staining followed by fluoremetric analysis (FIG. 6F). Notably, reprogramming without IWR1 did not show increase in mROS generation on d8, suggesting its role in mROS generation, alone or in combination (FIG. 6F). The inventors have also checked the number of Nrl-GFP⁺ reprogrammed cells in presence of different concentrations of mitotempo and found a dose dependent relationship (data not shown). These results confirmed that mROS play an important role in chemical reprogramming of photoreceptors.

To further confirm the involvement of mROS in chemical reprogramming, the inventors have used a genetic model of mROS generation. Depletion of Mitochondrial transcription factor (Tfam) has been shown to generate mROS (Vernochet et al., Cell metabolism 16:765-76, 2012). Earlier findings showed that IWR1 withdrawal reduced the reprogramming efficiency and mROS generation. So the inventors planned to use Tfam depleted MEFs for reprogramming in absence of IWR1. For knocking down of Tfam expression (FIG. 6C), Nrl-GFP MEFs were transduced with inducible lentivirus having Tfam shRNA (Dharmacon). Puromycin selected MEFS (for 3 days) were used for reprogramming by small molecules (w/o IWR1) with induction of Tfam shRNA starting on d3. Number of GFP positive converted cells was increased upon Tfam knockdown in absence of IWR1 compared to control wt Nrl-GFP positive cells (FIG. 6D and FIG. 6E). The inventors have confirmed the elevated level of mROS in Tfam knockdown cells by mitosox staining (FIG. 6F). These results clearly showed that mitochondrial ROS generated by small molecule treatment is a key player in photoreceptor reprogramming. The inventors have checked the possibility of mitochondrial ROS generation by other small molecules in the cocktail. There was no increase of mitochondrial ROS generation when the MEFs were treated with all small molecules alone or in combination (data not shown).

To check the possibility of NF-kB activation through mROS, luciferase activity was measured during chemical reprogramming of NF-KB-Luc-MEFs in presence of mitochondrial ROS scavenger. Interestingly mitotempo treatment (starting at d3) during reprogramming significantly decreases NF-kB activity from day 5 indicating that the activation is mROS dependent (FIG. 11A). To further confirm this observation Tfam knockdown MEFs that generates mROS were used for the chemical conversion in absence of IWR1. About a 2 fold increase of NF-kB activity was observed in Tfam knockdown cells compared to control on day 8 of reprogramming (FIG. 11A). Additionally the inventors have checked the binding of NF-kB at Ascl-1 locus upon treatment with mitotempo and found reduced binding (FIG. 6G). These results clearly demonstrated that mitochondrial ROS activated NF-kB binds to Ascl-1 regulatory regions and control its expression.

Small molecule induced stabilization and localization of Axin2 generates mROS. Next the mechanism of mROS generation during chemical reprogramming was investigated. One recent report suggested that simultaneous treatment of epiblast stem cells with CHIR and IWR1 causes stabilization of WNT signaling effector molecule axin2 (Kim et al., Nature communications 4:2403, 2013). Moreover HepG2 cells treated with XAV939 (a WNT signaling inhibitor functionally similar to IWR1) for several days can also stabilize the axin at cytosol. These overabundant axin molecules are targeted to the mitochondria and become associated with ETC complexIV (Shin et al., Experimental cell research 340:12-21, 2016). Association of axin with OXPHOS complexIV ultimately reduced the mitochondrial ATP production by hindering electron transport through repirosome. Furthermore exogenous Otx2 was found to be localized in mitochondria of retinal neurons and increases ATP production by interacting with F0/F1 ATPase. Considering these observations the inventors contemplated that reprogrammed cells generated by chemical cocktail (contains IWR1 and CHIR), might have stabilized axin2 at cytosol that subsequently targeted to mitochondria. This relocalization of axin2 to mitochondria could generate mROS by interacting with ETC supercomplexes which in turn activates NF-kB function. To test this hypothesis the inventors first checked the expression status of axin1 and axin2 in reprogramming intermediates, converted cells and MEFs. Intermediates and converted cells were found to have more stabilized axin2 compared to starting MEFs (FIG. 7A). No change in axin1 expression was evident between these cell types (data not shown). Subcellular localization of axin2 in reprogrammed cells has been tested by four color confocal microscopy. Z sectioning followed by 3D reconstruction showed that axin2 is localized to the mitochondria as evidenced by co-localization of axin2 with mitotracker (FIG. 7C). On the contrary axin2 was localized at cytosol of MEFs and no co-localization was found between mitotracker and axin2 (FIG. 7B). To further confirm the role of axin2 in mROS generation and reprogramming an axin2 depleted (inducible ShRNA purchased from Dharmacon) MEFs was generated and use for the chemical reprogramming. Converted intermediates generated from Axin2 knockdown MEFs (FIG. 7D) showed reduced ROS generation in presence of all small molecules compared to wild type Nrl-GFP MEFs (FIG. 7F). Furthermore axin2 depletion decreases the number of converted Nrl-GFP positive rod cells after chemical conversion (FIG. 7E). To further confirm the whole pathway the inventors measured NF-kB activation and Ascl1 expression in axin2 depleted reprogrammed cells on d8 of reprogramming. A decreased activity of NF-kB was observed and less expression of Ascl1 in reprogramming intermediates at d8 (FIG. 7G and FIG. 11B). These results clearly demonstrated that Axin2 is localized to the mitochondria in chemically converted cells and causes increased ROS generation from the organelle. Mitochondrial ROS in turn activated NF-kB which increased the expression of Ascl1. This NF-kB induced Ascl1 in turn control fibroblasts to photoreceptorconversion.

### B. Methods

Generation of chemically induced retinal neurons. All small molecules diluted in DMSO according to manufacturer's instruction. On day 0 about 35000 MEFs were seeded in each well of a 24 well plate (0.1% gelatin coated for O/N). At day 1 change the medium with photoreceptor induction medium (PIM) with V (0.5 mM), C (4.8 μM), R (2 μM) F (10 βM). On day 3 change the medium (PIM) with V, C, R, F plus IWR1 (I, 10 βM). Change the medium (PIM) with VCRFI on d5 and d6. On day 8 add Sonichedgehog (S, 3 nM), taurine molecules and factors. Finally on day 11 cells with GFP expression were observed. For the differentiation replace the medium with photoreceptor differentiation medium (PDM) and keep them up to d15 to d16. For Human Foreskin Fibroblasts (HFF-1) conversion all the protocol remains same except 5 μM IWR1 added on d3. S,T,R, BDNF, GDNF and NT3 added on day 5. Cells were fixed or analyzed on day 7.

Animal models and MEF isolation. All animals were breeding and handling was done according to the guidelines of Institutional Animal Ethics Committee. Nrl-GFP reporter mice line was a kind gift from Dr. Anand Swaroop from NEI. FSP1-Cre and fSftdTomato mice were purchased from Jackson lab (Barr Harbor, Mich., USA). MEFs were prepared from 12.5d embryo after removal of head limbs and tail according to a previously described protocol. For MEF preparation from FSP1-Cre and FsF-tdTomato mating, isolated embryos were checked under fluorescence microscope and red embryos were used for MEF isolation.

Transplantation of converted cells. After conversion in several 10 cm dishes cells were sorted based upon GFP expression. Then cells were suspended and kept in medium (IMR90) overnight. The next day cells were washed and suspended in PBS. 2 μl cell suspension (100K) was injected subretinally into the rd1 mice on d31. PBS injection was used as control. Cell and PBS transplanted eyes were analyzed for ERG on d45, d59 and d90.

Pupillometry. Dark adapted mice were used for capturing pictures under infrared illumination. Transplanted eyes were subjected to white light exposure through a light guide from 100-W goose arc lamp at an intensity of 60 Lux. Time lapse images were taken by an infrared camera (Sony, DCR-HC96). Pupil area for each mouse before and after light exposure was measured by ImageJ software. Change in pupil constriction was represented by difference in pupil area measured in dark and light to the pupil area in dark in each mouse.

QPCR and RT PCR. Total RNA was extracted by using a kit from Zymo Research (MicroPrep R1050). cDNA were prepared by High Capacity cDNA Reverse Transcription kit from Applied Biosystems according to the manufacturer's instructions. Isolated RNAs were treated with DNAaseI before c-DNA synthesis. RT-PCR and qPCR was performed by using specific primers. Thermal cycler from Applied Biosystems and OneStep Plus real time PCR were used for amplification. Results were normalized with Gapdh or HPRT. For list of primers see Table 1.

TABLE 1
List of Primers

| Spe- | | | | Pur- |
|---|---|---|---|---|
| cies | Gene | Forward | Reverse | pose |
| Mouse | eGFP | ATCATGGCCGACAAG | TCTCGTTGGGGTCTT | RT- |
| | | CAGAA | TGCTC | |
| | | (SEQ ID NO: 1) | (SEQ ID NO: 2) | PCR |
| Mouse | Nr1 | CCCGGGAGAGACAGG | CTCCAAACCGCCACA | RT- |
| | | AGCCC | CCCCC | |
| | | (SEQ ID NO: 3) | (SEQ ID NO: 4) | PCR |
| Mouse | Otx2 | GCCTCCAAACAACCT | GCTGGGCTCCAGATA | RT- |
| | | TAGCA | GACAC | |
| | | (SEQ ID NO: 5) | (SEQ ID NO: 6) | PCR |
| Mouse | Crx | ATCCAGGAGAGTCCC | GGCAGAGATGGGCTG | RT- |
| | | CATTT | TAAGA | |
| | | (SEQ ID NO: 7) | (SEQ ID NO: 8) | PCR |
| Mouse | Chx10 | GACTTCCCGGCTTCT | GGCACAGGAATCCAT | RT- |
| | | ACACA | TATGC | |
| | | (SEQ ID NO: 9) | (SEQ ID NO: 10) | PCR |
| Mouse | HPRT | TCAGTCAACGGGGGA | GGGGCTGTACTGCTT | RT- |
| | | CATAAA | AACCAG | |
| | | (SEQ ID NO: 11) | (SEQ ID NO: 12) | PCR |
| Mouse | Gapdh | AGGTCGGTGTGAACG | TGTAGACCATGTAGT | RT- |
| | | GATTTG | TGAGGTCA | PCR |
| | | (SEQ ID NO: 13) | (SEQ ID NO: 14) | |
| Mouse | Axin2 | TGACTCTCCTTCCAG | TGCCCACACTAGGCT | qRT- |
| | | ATCCCA | GACA | |
| | | (SEQ ID NO: 15) | (SEQ ID NO: 16) | PCR |
| Mouse | Asc11 | GCTGGCAGAAAGTCC | ATGCACATGGAGGCT | qRT- |
| | | GATTA | TCA | |
| | | (SEQ ID NO: 17) | (SEQ ID NO: 18) | PCR |
| Mouse | Tfam | ATTCCGAAGTGTTTT | TCTGAAAGTTTTGCA | qRT- |
| | | TCCAGCA | TCTGGGT | |
| | | (SEQ ID NO: 19) | (SEQ ID NO: 20) | PCR |
| Human | Recov- | CCTCTACGACGTGGA | GTGTTTTCATCGTCT | RT- |
| | erin | CGGTAA | GGAAGGA | PCR |
| | | (SEQ ID NO: 21) | (SEQ ID NO: 22) | |
| Human | Rdh12 | CTTCTCCCCCTTTGT | CTTTAGGGTTGGCCT | RT- |
| | | CAAGA | TCTCC | |
| | | (SEQ ID NO: 23) | (SEQ ID NO: 24) | PCR |
| Human | Rdk | GGACTGGTTCCTGGA | AAGCCAGGGTTCTCC | RT- |
| | | CTTCA | TCATT | |
| | | (SEQ ID NO: 25) | (SEQ ID NO: 26) | PCR |
| Human | Gapdh | GCTCAGACACCATGG | GTGGTGCAGGAGGCA | RT- |
| | | GGAAGGT | TTGCTGA | PCR |
| | | (SEQ ID NO: 27) | (SEQ ID NO: 28) | |
| Mouse | Asc11 | GCAACCGGGTCAAGT | GTCGTTGGAGTAGTT | ChIP |
| | | TGGT | GGGGG | |
| | | (SEQ ID NO: 29) | (SEQ ID NO: 30) | |

Immunohistochemistry and Immunoblotting. 4% paraformaldehyde was used to fix eyes. Cryo embedded eye samples were then sectioned in 14 μM thickness. Fixed eye sections were analyzed with primary and secondary antibodies listed in Table 2. 0.1% DAPI was used to stain the nucleus in mounting medium. Images were taken in Zeiss LSM510 confocal/Leica DMi8 florescence microscope. For immunoblotting total proteins were extracted by a commercially available lysis buffer (Thermo Scientific, #89900) and then concentration of proteins was measured with BCA protein assay kit (Thermo Scientific #23227). Equivalent amount of proteins were loaded into each well immunoblotted and antibody stained with standard procedure.

TABLE 2
Antibody list

| | | | Dilution | Amount | Dilution | |
|---|---|---|---|---|---|---|
| Antigen | Host | Company | for IF | for ChIP | for WB | Cat# |
| Axin2/conductin | Goat | Santa Cruz | 1:100 | | 1:200 | SC-8570 |
| Nrl | Mouse monoclonal | Santa Cruz | 1:100 | | | SC-398046 |
| Crx | Mouse monoclonal | Santa Cruz | 1:200 | | | SC-377138 |
| Beta actin | Mouse | Thermo Scientific | | | 1:2000 | MA5-15739 |
| GFP | Chicken | Abcam | 1:400 | | | Ab13970 |
| Axin1 | Goat | RD systems | | | 1:400 | AF3287 |
| Rhodopsin | Mouse | Millipore | 1:100 | | | MAB5316 |
| Recoverin | Rabbit | Abcam | 1:100 | | | Ab64945 |
| ChX10 | Sheep | Millipore | 1:100 | | | AB9016 |
| BrdU | Mouse | Thermo scientific | 1:500 | | | MA3071 |
| NF-kB | Mouse | Cell Signalling | | 6 μg | | 6956P |

### Electroretinogram

Immunofluorescence and Laser scanning confocal microscopy. For confocal microscopy converted and GFP sorted cells were seeded in a chambered coverglass (Nunc) and kept overnight. Next morning cells were stained with Mitotracker Red (500 nM) for 30 mins. Then cells were fixed in 4% PFA for 20 mins and stained with the axin2 Ab (Santa Cruz 1:100) overnight. Micrographs were taken in Zeiss LSM 510 confocal microscope. Data analysis and 3D reconstruction was done with the help of ZEN lite software. For immunofluorescence microscopy antibody stained cells were analyzed in a Leica fluorescence microscope. Alexa633, Alexa 549 and Alexa488 tagged secondary antibodies were used wherever necessary. For list of primary antibodies see Table 2

Measurement of mitochondrial ROS. Mitochondrial ROS was detected and quantified. Briefly GFP sorted cells or axin2 kd MEFS were seeded in a 96 well plate and incubated with Mitosox RED (500 nM) for 30 mins after or intermediary stage of chemical conversion. Cells were then washed twice with PBS and fluorescence has been monitored with a microplate reader set to 510 nm excitation (Ex bandwidth: 10 nm) and 595 nm emission (Em bandwidth 35 nm) wavelengths. mROS generation was also micrographed by Leica fluorescence microscope an quantified in Leica Application Suite X Software. At first background was subtracted and region of interest (ROI) has been drawn for each cells. Average intensity within each ROI was measured and exported to Excel data sheet. Average change in fluorescence was calculated for each type of cells. There were at least three replicates for each of the conditions.

FACS. For FACS sorting converted cells were passed through a 40 μm nylon cell stainer (Falcon) and suspended in PBS containing 3% serum. Starting Nrl-GFP MEFs were used as negative control. Cells were sorted in a BD LSRII Flow cytometer at Core facility of UT Southwestern Medical Center, Dallas. Sorted cells were collected in IMR90 medium, spun down and used for RNA extraction and other downstream applications.

RNAi and Generation of shRNA transduced MEFs. Lentiviral ShRNA constructs for axin2 (SMART vector inducible mouse shRNA, 12006) and Tfam (SMART vector inducible mouse shRNA, 21780) were purchased from Dharmacon. Lentiviral sups were collected for four days and concentrated by lenti-X concentartor (Clonetech). Aliquot of concentrated lentivirus was then used to transduce PO Nrl-GFP MEFs. Cells were selected for 3 days in presence of Puromycin (1 μg/ml). These cells were used for conversion experiments by small molecules.

Chromatin Immunoprecipitation and amplification. Real time PCR based quantitative ChIP analysis was performed according to kit from Millipore (17-295). Briefly 20000 cells were crosslinked with formaldehyde (1% final) for 10 mins at RT with gentle agitation. Sonicate the cells in such a way that the size of the chromatin would be between 300-500 bp. After preclearance with protein A agarose chromatins were used for immunoprecipitation with specific antibodies against NF-kB-p65. Immunoprecipitated chromatins were amplified by a whole genome amplification kit (WGA2) from Sigma. Amplified products were checked in agarose gel. Primers were designed to amplify 60-100 bp amplicons and were based on sequences in Ensembl Genome Browser for mouse. Products were amplified with a Fast SYBR Green Master Mix from Applied Biosystems in 20 μl reaction. The amount of product was determined relative to a standard curve of input chromatin. Dissociation curves showed single product for the amplicons. For Primers for ChIP analysis see Table 1.

Preparation of Nrl-DsRed promoter reporter construct. To prepare a Nrl-DsRed promoter reporter the promoter and reporter fragment were removed from a commercially available vector (pNrl-DsRed, Addgene plasmid #13764) by specific restriction enzymes. These vectors were then cloned into a gateway entry vector pENTR2B (ThermoFisher scientific A10463). Positive clones were then shuffled into a destination vector pLentiX1 Zeo DEST (Addgene plasmid #17299). This final product was then used for lentivirus preparation.

### Example 2

### Chemical Reprogramming of Human Adult Dermal Fibroblasts (HADF) to Photoreceptor (HCIPC)

### A. Results

Modified scheme for human adult dermal fibroblasts reprogramming is illustrated in FIG. 13A. qPCR analysis (fold change) of converted CiPCs from HADF shows increased expression of photoreceptor specific genes. A micrograph of Nrl stained HADF shows converted CiPCs. Comparison of conversion efficiency between earlier and modified conversion protocol. Expression of photoreceptor specific genes (Crx and Recoverin) in HADF (from a different source, Coriell Institute) converted CiPCs. Expression of photoreceptor specific genes (Nrl, Recoverin, Rhodopsin) in HADF (from ATCC) converted CiPCs (FIG. 13 and FIG. 14).

### B. Methods

Modified Protocol: D0: Human dermal fibroblasts are seeded (10⁶ cells/well) on 6 well plate coated with 0.1% gelatin (O/N) in IMR-90 medium. D1: Medium changed with photoreceptor induction medium (PIM) having Valproic acid (V, 0.5 mM), CHIR (C, 3 μM), Repsox (R, 1 μM), Forskolin (F, 10 μM), IWR1 (I, 10 μM). D3: Medium change with VCRFI. D5: Medium change with VCRFI. D7: Medium change with VCRFI. D9: Medium change with VCRFI. Analyze cells for gene expression. D10: Harvest cells.

Photoreceptor Induction Medium (PIM) includes DMEM/F12 containing KO serum 5 ml, B27 1 ml, Noggin 12.5 μl, IGF1 1.25 μl. Make the final volume up to 50 ml.

### Example 3

### Chemical Reprogramming Mouse Muller Glial Cells to Retinal Neurons

FIG. 15 shows conversion of mouse primary muller cells to retinal ganglion cells. FIG. 15A shows Muller cells before conversion. FIG. 15B shows CiRGC cells after conversion on day 3 by chemical cocktail. FIG. 15C shows real time qPCR analysis showing expression of RGC specific genes like Brn3a, Brn3b, Isl1, Nefl and NeN.

Modified protocol includes: D0: Mouse primary muller cells are seeded on a 6 well plate coated (O/N) with 0.1% gelatin in medium containing DMEM and 10% FBS (seeding density, 10⁶ cells/well). D1: Add IFV in PIM (photoreceptor induction medium, concentrations same as above). D2-D4: Retinal ganglion cells appeared.

### Example 4

### Chemical Reprogramming Human Muller Glial Cells to Retinal Neurons

FIG. 16 illustrates conversion of human primary muller cells to retinal ganglion cells (hCiRGC). FIG. 16A shows human muller cells before conversion. FIG. 16B shows hCiRGC cells after conversion on day 3 by chemical cocktail. FIG. 16C shows real time qPCR analysis showing expression of RGC specific genes like Brn3a, Brn3b, Isl1, Nefl and NeN.

The modified protocol includes: D0: Muller cells are seeded on in a 6 well (0.1% gelatin coated for O/N) plate in medium containing DMEM and 10% FBS (10⁶ cells/well). D1: Add VCRFI in PIM (photoreceptor induction medium). D2-D4: Retinal ganglion cells appeared.

### Example 5

### Chemical Reprogramming of Embryonic Stem (ES) Cells to Retinal Neurons

FIG. 17 shows conversion of ES cells to neuron like cells by chemicals (A) Mouse ES cells on day1 before chemical treatment. (B) ES cell derived chemically converted neuron like cells on d6. (C) ES cell derived chemically converted neuron like cells on d7.

The same protocol as mouse embryonic fibroblasts to photoreceptor and RGC cell conversion was used. The protocol included: D0: Mouse ES cells seeded in 6 well plate. D1: Medium change with VCRF containing PIM. D3: Medium change with VCRFI containing PIM. D5: Medium change with VCRFI. D6-D7: neuron shaped cells that resemble photoreceptor and ganglion cells appeared.

1.-52. (canceled)

53. A method of chemically converting a somatic cell to a target cell, the method comprising culturing the somatic cell in the presence of reprogramming agents converting the somatic cell into a target cell, the reprogramming agents comprising a first reprogramming composition comprising (i) epigenetic modifier, wherein the epigenetic modifier is a Cytochrome P450 2C9 (CYP2C9) inhibitor, (ii) glycogen synthase kinase-3 (GSK-3) inhibitor/WNT agonist, (iii) TGFβR/ALK5 inhibitor, (iv) adenyl cyclase activator, (v) WNT inhibitor, the culturing forming a reprogrammed cell culture

54. The method of claim 53, further comprising culturing the somatic cell in the presence of a second reprogramming composition comprising enhancing agents sonic hedgehog (S), taurine (T), and/or retinoic acid (R).

55. The method of claim 53, wherein the Cytochrome P450 2C9 (CYP2C9) inhibitor is valproic acid.

56. The method of claim 53, wherein the Glycogen synthase kinase-3 (GSK-3) inhibitor/WNT agonist is CHIR99021.

57. The method of claim 53, wherein the TGFβR/ALK5 inhibitor is Repsox.

58. The method of claim 53, wherein the adenyl cyclase activator is forskolin.

59. The method of claim 53, wherein the WNT inhibitor is IWR-1 or XAV939.

60. The method of claim 53, wherein the somatic cell is a fibroblast, monocyte, epithelial cell, cells isolated from the blood, skin fibroblast, keratinocytes, or urine-derived epithelial cells.

61. The method of claim 53, wherein Axin2 stabilization or accumulation results in generation of a target cell.

62. The method of claim 53, wherein the target cell is a hepatocyte, cardiomyocyte, sensory hair cell, retinal photoreceptor, retinal ganglion cell, retinal progenitor cell, or a retinal pigment epithelial cell.

63. The method of claim 53, wherein the somatic cell is harvested from a subject, reprogrammed into a target cell, and the target cell is treated with a potential therapeutic agent to determine the effectiveness of the agent to improve cell health or reduce cell death in the subject.

64. The method of claim 53, wherein the somatic cell or target cell is manipulated through gene editing to silence or repair a defective gene.

65. The method of claim 53, further comprising delivering a the target cell to subject in need thereof.

66. The method of claim 65, wherein the subject has retinal atrophy, optic nerve injury, optic nerve atrophy, age-related macular degeneration, inherited retinal degeneration, diabetic retinopathy, sickle cell retinopathy, glaucoma, cystoid macular edema, retinal detachment, vascular occlusion, photoreceptor cell degeneration, infection, vision loss, or any combination thereof.

67. A hepatocyte, cardiomyocyte, or retinal cell produced by the method of claim 53.

68. The retinal cell of claim 67, wherein the retinal cell is photoreceptor-like, RGC-like cell, retinal progenitor cell-like or a retinal pigment epithelial cell-like cell.

69. A method of treating a disorder of the eye to improve function of a cell resident in the retina or produce new retinal cells in a subject in need thereof comprising delivering to the eye of the subject an effective amount of a combination of (i) epigenetic modifier, (ii) glycogen synthase kinase-3 (GSK-3) inhibitor/WNT agonist, (iii) TGFβR/ALK-5 inhibitor, (iv) cAMP raising compound, (v) WNT antagonist and (vi) enhancing agents sonic hedgehog (S), taurine (T), and/or retinoic acid (R).

70. The method of claim 69, wherein the cell resident in the retina is a glial cell or neuron.

71. The method of claim 69, wherein all or a combination of the small molecules (i), (ii), (iii), (iv), (v), and (vi) is administered to rejuvenate or restore the function of damaged, diseased, and/or aged cells.

72. The method of claim 69, wherein the disorder includes retinal atrophy, optic nerve injury, optic nerve atrophy, age-related macular degeneration, inherited retinal degeneration, diabetic retinopathy, sickle cell retinopathy, glaucoma, cystoid macular edema, retinal detachment, vascular occlusion, photoreceptor cell degeneration, infection, vision loss and any combination thereof.


SEQUENCE LISTING (30 sequences)

SEQ ID NO: 1 - DNA, 20 bp, Artificial Sequence
  synthetic oligonucleotide
atcatggccgacaagcagaa

SEQ ID NO: 2 - DNA, 20 bp, Artificial Sequence
  suynthetic oligonucleotide
tctcgttggggtctttgctc

SEQ ID NO: 3 - DNA, 20 bp, Artificial Sequence
  synthetic oligonucleotide
cccgggagagacaggagccc

SEQ ID NO: 4 - DNA, 20 bp, Artificial Sequence
  synthetic oligonucleotide
ctccaaaccgccacaccccc

SEQ ID NO: 5 - DNA, 20 bp, Artificial Sequence
  synthetic oligonucleotide
gcctccaaacaaccttagca

SEQ ID NO: 6 - DNA, 20 bp, Artificial Sequence
  synthetic oligonucleotide
gctgggctccagatagacac

SEQ ID NO: 7 - DNA, 20 bp, Artificial Sequence
  synthetic oligonucleotide
atccaggagagtccccattt

SEQ ID NO: 8 - DNA, 20 bp, Artificial Sequence
  synthetic oligonucleotide
ggcagagatgggctgtaaga

SEQ ID NO: 9 - DNA, 20 bp, Artificial Sequence
  synthetic oligonucleotide
gacttcccggcttctacaca

SEQ ID NO: 10 - DNA, 20 bp, Artificial Sequence
  synthetic oligonucleotide
ggcacaggaatccattatgc

SEQ ID NO: 11 - DNA, 21 bp, Artificial Sequence
  synthetic oligonuceotide
tcagtcaacgggggacataaa

SEQ ID NO: 12 - DNA, 21 bp, Artificial Sequence
  synthetic oligonucleotide
ggggctgtactgcttaaccag

SEQ ID NO: 13 - DNA, 21 bp, Artificial Sequence
  synthetic oligonucleotide
aggtcggtgtgaacggatttg

SEQ ID NO: 14 - DNA, 23 bp, Artificial Sequence
  synthetic oligonucleotide
tgtagaccatgtagttgaggtca

SEQ ID NO: 15 - DNA, 21 bp, Artificial Sequence
  synthetic oligonucleotide
tgactctccttccagatccca

SEQ ID NO: 16 - DNA, 19 bp, Artificial Sequence
  synthetic oligonucleotide
tgcccacactaggctgaca

SEQ ID NO: 17 - DNA, 20 bp, Artificial Sequence
  synthetic oligonucleotide
gctggcagaaagtccgatta

SEQ ID NO: 18 - DNA, 18 bp, Artificial Sequence
  synthetic oligonucleotide
atgcacatggaggcttca

SEQ ID NO: 19 - DNA, 22 bp, Artificial Sequence
  synthetic oligonucleotide
attccgaagtgtttttccagca

SEQ ID NO: 20 - DNA, 22 bp, Artificial Sequence
  synthetic oligonucleotide
tctgaaagttttgcatctgggt

SEQ ID NO: 21 - DNA, 21 bp, Artificial Sequence
  synthetic oligonucleotide
cctctacgacgtggacggtaa

SEQ ID NO: 22 - DNA, 22 bp, Artificial Sequence
  synthetic oligonucleotide
gtgttttcatcgtctggaagga

SEQ ID NO: 23 - DNA, 20 bp, Artificial Sequence
  synthetic oligonucleotide
cttctccccctttgtcaaga

SEQ ID NO: 24 - DNA, 20 bp, Artificial Sequence
  synthetic oligonucleotide
ctttagggttggccttctcc

SEQ ID NO: 25 - DNA, 20 bp, Artificial Sequence
  synthetic oligonucleotde
ggactggttcctggacttca

SEQ ID NO: 26 - DNA, 20 bp, Artificial Sequence
  synthetic oligonucleotide
aagccagggttctcctcatt

SEQ ID NO: 27 - DNA, 22 bp, Artificial Sequence
  synthetic oligonucleotide
gctcagacaccatggggaaggt

SEQ ID NO: 28 - DNA, 22 bp, Artificial Sequence
  synthetic oligonucleotide
gtggtgcaggaggcattgctga

SEQ ID NO: 29 - DNA, 19 bp, Artificial Sequence
  synthetic oligonucleotide
gcaaccgggtcaagttggt

SEQ ID NO: 30 - DNA, 20 bp, Artificial Sequence
  synthetic oligonucleotide
gtcgttggagtagttggggg